(19) United States
(12) Patent Application Publication
(10) Pub. No.: US 2002/0002140 A1
(43) Pub. Date: Jan. 3, 2002

(54) Novel bisphosphonates and uses thereof

(76) Inventors: Michael F. Holick, Sudbury, MA (US); Halasya Ramanathan, Worcester, MA (US); George M. Blackburn, Sheffield (GB)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 09/760,382

(22) Filed: Jan. 16, 2001

Related U.S. Application Data
(60) Provisional application No. 60/176,006, filed on Jan. 14, 2000.
(60) Provisional application No. 60/210,021, filed on Jun. 8, 2000.

(51) Int. Cl. A61K 31/70; C07H 5/06

(52) U.S. Cl. 514/25; 536/18.7

(57) ABSTRACT

Glycosides and orthoester glyco side derivatives of bisphosphonate compounds useful for treating and/or preventing hypercalcaemia of malignancy, Paget's disease, osteoporosis, metastatic cancer in bone and soft tissue and periodontal disease have markedly enhanced intestinal absorption and enhanced bioavailability.

### CROSS REFERENCE TO RELATED APPLICATIONS

This claims priority to U.S. Provisional Application Nos. 60/176,006, filed Jan. 14, 2000, and 60/210,021, filed Jun. 8, 2000, the contents of each of which are fully incorporated by reference herein.

### BACKGROUND OF THE INVENTION

1. Field of the Invention

The present invention relates to novel derivatives of bisphosphonate compounds, and to their use in the treatment and/or prevention of such conditions as hypercalcaemia of malignancy, Paget's disease, osteoporosis, metastatic cancer in bone and soft tissue, and periodontal disease.

2. Related Art

Bisphosphonates are synthetic compounds, structurally related to pyrophosphate, in that two phosphonates are attached to a common atom. More specifically, bisphosphonates of particular interest contain a P—C—P bond, whereas pyrophosphate has a P—O—P bond. The difference in the two chemical bonding schemes means that bisphosphonates are resistant to enzymatic hydrolysis in osseous tissue, for example.

Bisphosphonates are potent inhibitors of bone resorption and have been successfully used in the treatment and/or prevention of hypercalcaemia associated with periodontal disease, osteoporosis, bone malignancy and metastatic cancer of the bone. In addition, bisphosphonates have been successfully used in the treatment and/or prevention of conditions which involve abnormal calcium and phosphate metabolism, such as Paget's disease.

Bisphosphonates are also used to treat other more rare disorders such as mastocytosis, fibrous displasia, Gaucher's disease and osteogenesis imperfecta.

Bisphosphonates have further been used to inhibit mineralisation in the treatment of heterotropic ossification and myositis ossificans progressiva. In addition, bisphosphonates have found use in the stabilization of prostheses such as in total hip replacement.

U.S. Pat. No. 5,403,829 teaches that the main effect of the bisphosphonates is their ability to inhibit bone resorption. Contrary to the effect on mineralisation, the mechanism involved in bone resorption is cellular (c.f. Fleisch, Drugs 42: 919-44 (1991)). These different effects vary greatly according to the structure of the individual bisphosphonate compound.

The half-life of circulating bisphosphonates is very short; in the order of minutes to hours. Of any a given bisphosphonate dose, 20 to 50% is taken up by the skeleton, while the rest is excreted in the urine. The half-life in bone, however, is far longer and depends upon the turnover rate of the skeleton itself.

U.S. Pat. No. 5,403,829 further teaches that of the many compounds belonging to the bisphosphonate family, clodronate has been widely used in hypercalcaemia and osteolysis of malignancy (c.f. Bonjour et al., Calcif. Tissue Int. 46 Suppl. 20-25 (1990)). All published reports indicate that clodronate can normalize plasma calcium in the majority of hypercalcaemic, rehydrated cancer patients in whom increased bone resorption is the prevailing disturbed calcium flux (Fleisch, supra).

Various phosphonate compounds are also reported in the patent literature as being useful in the treatment and/or prevention of a variety of bone diseases. U.S. Pat. Nos. 5,753,634 and 5,763,611 disclose cyclic bisphosphonate compounds and methods for treating or preventing pathological conditions characterized by abnormal calcium and phosphate metabolism (e.g., Paget's disease), respectively.

U.S. Pat. No. 5,409,91 1 discloses prostaglandin-bisphosphonate compounds that are effective as delivery agents of prostaglandins to treat osteoporosis and related bone diseases. The compounds also simultaneously deliver a bisphosphonate which inhibits bone resorption.

U.S. Pat. No. 5,428,181 discloses bisphosphonates that show significant therapeutic effects on bone diseases such as osteoporosis, rheumatoid arthritis and osteoarthritis.

U.S. Pat. No. 5,668,120 discloses methods of inhibiting alveolar bone resorption or the undesirable movement of teeth of a human or other animal. The invention relates to iontophoretic delivery of a bisphosphonic acid compound or pharmaceutically acceptable salt and ester thereof, to the oral tissue.

Other pharmaceutically active bisphosphonates are disclosed in U.S. Pat. Nos. 4,810,486; 5,103,036; 5,157,027; 5,183,815; 5,196,409; 5,312,954; 5,317,015; 5,360,797; 5,602,115; 5,616,571; 5,635,495; 5,753,633 and 5,856,314.

It is well known that bisphosphonates have very poor bioavailability when given orally. For example, in the case of FOSAMAX (alendronate sodium; Merck), only about 0.7% of the drug is absorbed from the gut, on average. In addition, the bisphosphonates are also associated with cellular toxicity. There have been reports of oral alendronate giving rise to esophageal irritation and ulceration, and it is generally not possible to administer pamidronate (a homologue of alendronate) orally, as the cytotoxicity is considerably worse.

### SUMMARY OF THE INVENTION

However, we have now, surprisingly, found that the glycoside and orthoester glycoside derivatives of bisphosphonate compounds have markedly enhanced intestinal absorption. They have, thus, enhanced bioavailability, enabling them to be given orally, and substantially reducing the amount of compound needed to be administered and/or reducing the amount of time elapsed before a therapeutic effect occurs. In addition, at effective concentrations, the cytotoxicity of these compounds is substantially reduced.

Thus, in a first aspect, the present invention provides a glycoside or orthoester glycoside derivative of a therapeutically useful bisphosphonate compound, or pro-drug thereof, preferably wherein the bisphosphonate compound is useful in the treatment and/or prevention of hypercalcaemia or osteoporosis.

### BRIEF DESCRIPTION OF THE FIGURES

FIG. 1 shows a comparison between a glycosylated compound of the invention (the glycosylated product -1-((4-O-glucopyranosyl)oxyphenyl)-aminoethylidene-1,1-bisphosphonic acid) and a non-glycosylated compound (1-ethylidene-1,1-bisphosphonic acid).

FIGS. 2 and 3 compare the effects of glycosylated and non-glycosylated alendronate.

FIG. 2 shows that both the alendronate and its glucoside have similar effects on bone resorption.

FIG. 3 shows that the alendronate glycoside exhibits substantially less toxicity than unglycosylated alendronate.

### DETAILED DESCRIPTION OF THE PREFERRED EMBODIMENTS

It will be appreciated that any therapeutically active bisphosphonate compound, or pro-drug thereof, may be derivatized to form a compound of the invention. Indeed, not only can those compounds currently prescribed be prepared as glycosides or orthoester glycosides (which terms are used interchangeably herein, except as otherwise indicated, or apparent), but also those compounds which have not been prescribable, at least orally, owing to either low bioavailability and/or cytotoxicity when given orally.

It will be appreciated that the term “therapeutically useful” does not exclude compounds which cannot currently be prescribed because of bioavailability problems.

In a preferred aspect, the present invention provides a compound of formula (I):

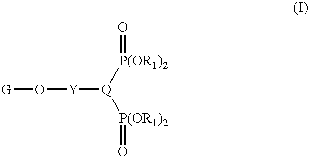

wherein G is a straight or branched chain glycosidic residue containing 1-20 glycosidic units per residue, or G is an orthoester glycoside moiety of the Formula (II):

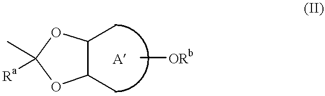

wherein A′ is a glycofuranosyl or glycopyranosyl ring;

R^{a} is hydrogen;

R^{b} is hydrogen or a straight or branched chain glycosidic residue containing 1-20 glycosidic units per residue;

each R₁, which may be the same or different, is hydrogen, alkyl, aryl, benzyl or alkali metal cation, or two —OR₁, groups, on the same phosphorus atom, taken together with —(CH₂)₂— —(CH₂)₃—, or —CH₂C(CH₃)₂CH₂—, form a heterocyclic ring containing one phosphorus, two oxygens and two or three carbons;

Q is selected from the group consisting of:

(1) —CH—, —CNH₂—, —COH—, —CCl—, —CF— or —C—alkyl—;

(2) —C(R₄)(R₅)(CH₂)ₘ[C(R₆)(R₇)]ₙ—;

wherein n is 0 or 1 and m is an integer from 1 to 8;

R₄ and R₆, which may be the same or different, are hydrogen, —SO₃H, a lower aliphatic group which may optionally contain one or more heteroatoms and which contains at least one —SO₃H group or a covalent bond to Y,

R₅ and R₇, which may be the same or different, are hydrogen, —OH, —NH₂, —NHMe, —NMe₂, —SO₃H, substituted alkyl or a covalent bond to Y; or

R₄ and R₅ taken together with the atom to which they are bound form a carbonyl, thiocarbonyl, or —NOH; and

R₆ and R₇ taken together with the atom to which they are bound form a carbonyl, thiocarbonyl, or ═NOH;

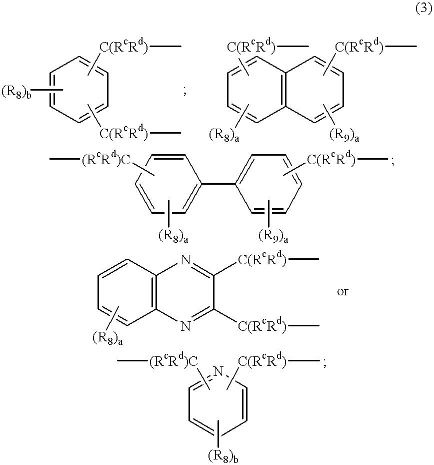

wherein R₈, and R₉, which may be the same or different, are:

(a) a covalent bond to Y, —NO₂ or —NH₂;

(b) —SR₁₁ wherein R₁₁ is hydrogen, alkyl, phenyl, acyl, benzoyl, aralkyl, halogen, allyl or a covalent bond to Y;

(c) —OR₁₀ wherein R₁₀ is hydrogen, alkyl, allyl, acyl, benzoyl, aralkyl or a covalent bond to Y;

(d) —CH₂)ₚCO₂A′, wherein A′ is hydrogen, alkyl or a covalent bond to Y;

(e) —CH₂)ₚCH₂OR₁₀ wherein R₁₀ is defined as above;

(f) —CH₂NH wherein R₁₂ is hydrogen, alkyl, phenyl, acyl, benzoyl, aralkyl or a covalent bond to Y;

(g) —CH₂N(R₁₂)(R₁₃) wherein R₁₂ and R₁₃ can be the same or different and are hydrogen, alkyl, phenyl, acyl, benzoyl, aralkyl or a covalent bond to Y;

is 1 or 2;

b is 1 to 4;

R^{c} and R^{d}, which may be the same or different in each instance, are hydrogen or alkyl;

with the proviso that when the ring containing R₈ is a pyridine ring, b is 1 to 3; p is to 5;

with the proviso that only one of A′, R₄, R₅, R₆, R₇, R₈, R₉, R₁₀, R₁₁, R₁₂ and R₁₃ is a covalent bond to Y;

Y is chosen from the group consisting of optionally substituted C₁₋₁₀ alkylene, aryl, heteroaryl, heterocyclo, a steroidal hormone, a compound exhibiting oestrogenic activity or a prostaglandin; and pharmaceutically acceptable salts or esters thereof.

It is generally preferred that the compounds of the present invention possess a P—C—P linkage, in order to best mimic pyrophosphates.

Thus, in a alternative preferred aspect, there is provided a compound of Formula (I′):

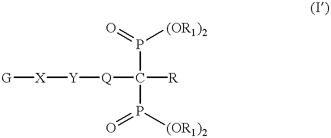

wherein each OR₁ is the same or different and is OH or a hydrolysable group, or two OR₁ groups on the same phosphorus atom form a substituted or unsubstituted 5, 6 or 7 membered heterocyclic ring;

R is H, alkyl or halogen or is a group —O—G, —S—G, —NH—G or —NR₁₂—G; each G is the same or different and is hydrogen or a straight or branched chain glycosidic residue or glycosidic orthoester residue, amino derivative or sulfate thereof, provided that at least one group G is a glycosidic residue or glycosidic orthoester residue,

X is O, S, NH or NR₂;

each R₁₂ is the same or different and is hydrogen, alkyl, phenyl, acyl, benzoyl or aralkyl;

Q is an optionally substituted alkylene or alkenylene group or is an optionally substituted alkylene containing at least one O, S or NH or is O, S or NH, or is a direct bond to Y;

Y represents a binary or tertiary alkyl-substituted amine, C₁₋₁₀ alkylene, aryl, heteroaryl, heterocyclyl, a steroidal hormone, a group exhibiting oestrogenic activity or a prostaglandin, said group Y being optionally substituted; provided that, in —X—Y—Q— there is no direct bond between one O, S or N atom and another O, S or N,

and pharmaceutically acceptable salts, esters and pro-drugs thereof.

When OR, is a hydrolysable group, then R₁ may conveniently represent: alkyl, for example methyl, ethyl, propyl, t-butyl; aryl, for example phenyl; aralkyl, for example benzyl; any of which may be optionally substituted, for example by lower alkyl or halogen; or R₁ may represent a suitable cation, such as ammonium or substituted ammonium, or an alkali metal cation, for example sodium or potassium.

Where two OR₁ groups on the same phosphorus atom form a substituted or unsubstituted 5, 6 or 7 membered heterocyclic ring, then it will be appreciated that phosphorus will be one member of the ring, together with two oxygens, the two R₁ groups together representing, for example, an ethylenic or propylenic linkage which optionally be substituted by one or more halogen atoms or methyl or ethyl groups.

When R₁ represents an alkyl group, then this may conveniently be a C₁₋₆ alkyl, such as methyl, ethyl, propyl, butyl, methylpropyl, t-butyl, pentyl, dimethylpropyl, hexyl, dimethylbutyl or ethylbutyl. Where alkyl groups are mentioned herein, then they are generally preferably as exemplified above.

In the case of R, preferred alkyl groups contain 1 or 2 carbon atoms. Methyl and ethyl groups are particularly preferred, especially methyl.

When R is halogen and, in general, when halogen is specified herein, then this is usefully chloro, fluoro, bromo or iodo, especially chloro.

When R contains a glycoside or orthoester glycoside, then this is generally as exemplified below, and the group is preferably an —O—G or —N—G group.

Where G represents a glycosidic residue, then it is preferred that it contain 1-20 glycosidic units. When G is a glycosidic orthoester residue, then it is preferred that it have the Formula (II):

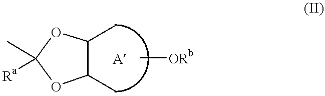

wherein A′ represents a glycofuranosyl or glycopyranosyl ring or amino derivative thereof;

R^{a} is hydrogen, Cl₁₋₄ alkyl, C₇₋₁₀ aralkyl, phenyl; or phenyl substituted by chloro, fluoro, bromo, iodo, C₁₋₄ alkyl or C₁₋₄ alkoxy; or naphthyl; and

R^{b} is hydrogen or a straight or branched chain glycosidic residue containing 1-20 glycosidic units per residue.

It is preferred that G has less than 10 and, more preferably, 3 or less glycosidic units. Specific examples are those containing 1 or 2 glycosidic units in the glycoside residue, such as glucose and sucrose, with one being most preferred.

By glycosidic units are meant glycopyranosyl or glycofuranosyl, as well as their sulfates, amino sugar and/or deoxy derivatives. The configuration of each unit may be D or L, although D is generally preferred. The residues may be homopolymers, random or alternating polymers, or block copolymers of these monomers.

The glycosidic units have free hydroxy groups, or the hydroxy groups may be acylated, e.g. with a group R⁵—(C═O)—, wherein R⁵ is hydrogen, C₁₋₆ alkyl, C₆₋₁₀ substituted or unsubstituted aryl or C₇₋₁₆ aralkyl. Preferably, the acyl groups are acetyl or propionyl. Other preferred R⁵ groups are phenyl, nitrophenyl, halophenyl, lower alkyl substituted phenyl, lower alkoxy substituted phenyl and the like or benzyl, lower alkoxy substituted benzyl and the like.

The glycopyranose or glycofuranose ring or amino derivative thereof may be fully or partially acylated or completely deacylated. The completely or partially acylated glycoside is useful as a defined intermediate for the synthesis of the deacylated material. Useful protecting groups include, but are not limited to, acetyl, benzoyl, nicotinoyl, benzyl, methyl and phenyl.

Among the possible glycopyranosyl structures are glucose, mannose, galactose, gulose, allose, altrose, idose, or talose. Among the furanosyl structures, the preferred ones are derived from fructose, ribose, arabinose or xylose. Among preferred diglycosides are sucrose, cellobiose, maltose, lactose, trehalose, gentiobiose, and melibiose. Among the triglycosides, the preferred ones may be raffinose or gentianose.

Preferred aminosugar derivatives are N-acetyl-D-galactosamine, N-acetyl-D-glucosamine, N-acetyl-D-mannosamine, N-acetylneuraminic acid, D-glucosamine, D-lyxosylamine, D-galactosamine, chondroitin, and the like. In addition, such active units as chondroitin sulfate and D-glucosamine sulfate may also be employed, as such sub-units independently have advantageous therapeutic osteopathic properties, and can enhance or complement the activity of the bisphosphonate compound.

Where there are linked glycosidic units, i. e., there is a di or polyglycosidic residue, the individual glycosidic rings may be bonded by 1- 1, 1-2, 1-3, 1-4, 1-5 or 1-6 bonds, most preferably 1-2, 1-4 and 1-6. The linkages between individual glycosidic rings may be α or β.

It is generally preferred that only one group G is a glycosidic or glycosidic orthoester group.

When Q is an alkylene group, then this preferably contains up to 7 carbons in the chain, with methylene, ethylene, propylene, butylene and pentylene being preferred. These may be substituted, if desired, preferably with methyl, ethyl or halogen.

In general, where a group is optionally substituted then unless otherwise specified or apparent, then it is preferred that the substituent not sterically hinder any other part of the molecule, especially the bisphosphonate moiety, and be generally non-reactive.

When Q is an alkenylene group, then this and any substituents are generally as described for alkylene, above. While it is possible and envisaged herein, for the alkenylene group to have two or more double bonds, it is preferred that it only have one.

Q may be, or contain, O, S or NH. It is generally preferred that there be no more than two of these chain members and that, where there are two, then they be spaced by at least a methylene group. Preferably, however, Q only contains one of O, S and NH. Any such O, S or NH may be linked directly to the methylenic carbon of the bisphosphonate group, or may be spaced apart therefrom by one or more carbon atoms.

Preferred meanings for Q are, O, S, NH and unsubstituted alkylene.

When Y represents an amine, then the nitrogen is preferably directly linked to Q. Where there are two alkyl substituents, then these may, optionally, form a heterocyclyl group with the nitrogen to which they are attached. The or each alkyl substituent may be substituted by, for example, halogen, cycloalkyl, aryl, aralkyl, heteroaryl and heteroaralkyl groups. Preferably the alkyl group or groups are unsubstituted or are substituted by one or two halogen atoms.

When Y is C₁₋₁₀ alkylene, then it is preferably as defined above, or is a cycloalkyl group containing 3 to 7 carbons. Preferred alkylene groups contain 1 to 4 carbon atoms, more preferably 1 or 2.

When Y is aryl, then this will generally contain between 6 and 14 ring members and may be substituted by such groups as OH, halo, NH and alkyl, with halogen and alkyl being preferred, where there is a substituent. Preferred examples of aryl groups are phenyl, chlorophenyl and naphthyl.

Where Y represents a heteroaryl group, then this will generally contain between 5 and 10 ring members, of which one, two or three may be selected from N, S and O. In general, preferred heteroatoms are N, and exemplary heteroaryls include pyrazolyl, imidazolyl and pyridinyl. Where such groups are substituted, then this may be as exemplified above for aryl.

When Y is a heterocyclic group, then this may generally be as exemplified for heteroaryl, provided that the ring is not completely unsaturated. Substituents may be as exemplified above, and suitable examples of heterocyclic groups include pyrrolidinyl and pyrimidinyl.

Where Y represents a steroidal hormone, a group exhibiting oestrogenic activity or a prostaglandin, then these may generally be as exemplified below.

Preferred meanings for Y are alkyl, tertiary amine, aryl, heteroaryl, cycloalkyl, heterocyclyl, optionally further substituted by methyl or chloro groups.

Particular examples of Y are chosen from the group consisting of C₁₋₁₀ alkylene, aryl, heteroaryl, heterocyclyl, a steroidal hormone, a compound exhibiting oestrogenic activity or a prostaglandin, any of which may be optionally substituted with alkyl, alkoxy, substituted alkoxy, acyl, amino, aryl, substituted aryl, aryloxy, substituted aryloxy, cyano, halogen, hydroxyl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxyl-heteroaryl, carboxyl-substituted heteroaryl, carboxylheterocyclic, carboxyl-substituted heterocyclic, cycloalkyl, substituted cycloalkyl, -SH, thioalkyl, substituted thioalkyl, thioaryl, substituted-thioaryl, thiocycloalkyl, substituted-thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy or substituted cycloalkoxy.

In a preferred embodiment, at least 3 R₁ groups are hydrogen atoms.

By “pharmaceutically acceptable” is generally meant that administration of the compound to a patient is, on balance, therapeutically advantageous.

Suitable salts are organic and inorganic acid salts, and are exemplified below. Likewise, esters may be organic or inorganic.

Pro-drugs may be any suitable derivative of a compound of the invention which breaks down in vivo to yield a suitable bisphosphonate or glycoside or orthoester glycoside thereof. In this respect, it will be appreciated that the glycosylated compounds of the invention may frequently, in and of themselves, be pro-drugs, in that the glycosyl group will frequently be deleted in vivo to yield the parent compound. However, we also demonstrate herein that the compounds of the invention appear to be active without having to be cleaved.

Individually preferred bisphosphonate compounds which, glycosylated, form a part of the invention, include: alendronate (4-amino-1-hydroxybutylidene)bisphosphonate; EB-1053 (1-hydroxy-3-(1-pyrrolidinyl)propylidene)bisphosphonate; etidronate (1-hydroxyethylidene)bisphosphonate; ibandronate (1-hydroxy-3-(methylpentylamino)propylidene)bisphosphonate; incadronate ((cycloheptylamino)methylene)bisphosphonate; neridronate (6-amino-1-hydroxyhexylidene)bisphosphonate; olpadronate (3-(dimethylamino)-1-hydroxypropylidene)bisphosphonate; pamidronate (3-amino-1-hydroxypropylidene)bisphosphonate; risedronate (1-hydroxy-2-(3-pyridinyl)ethylidene)bisphosphonate; riludronate ((4-chlorophenylthio)methylene)bisphosphonate; YH 529 (1-hydroxy-2-imidazo(3,2a)pyridin-3-ylethylidene)bisphosphonate; and zoledronate (1-hydroxy-2-(1H-imidazol-1-yl)ethylidene)bisphosphonate.

Where possible, it is preferred that the above compounds are derivatized on a free OH, NH₂ or SH grouping. Where such a grouping is not available, or where it is desired to provide a glycoside elsewhere, then it is preferred to further substitute the compound with an OH or NH group which may then be derivatized. Such additional groups are preferred to be distal from the bisphosphonate moiety.

The invention further provides a pharmaceutical composition comprising a compound of the present invention and a pharmaceutically acceptable carrier therefor.

There is further provided one or more compounds of the present invention for use in the treatment and/or prophylaxis of a condition susceptible of treatment by bisphosphonates, especially those indicated herein.

In addition, there is provided a method of treatment of a condition treatable by administration of a bisphosphonate compound, comprising administration of a non-toxic, efficacious amount of a compound of the present invention to a patient in need thereof. In this context, non-toxic has the same meaning as the term “pharmaceutically acceptable”.

The preferred route of administration is per os or by transdermal patch, as the glycosides and orthoester glycosides of the present invention also have enhanced skin penetration properties over the underivatized compounds. It will be appreciated that, while the present invention extends to all suitable derivatized bisphosphonates, those compounds formulated for pharmaceutical use should be pharmaceutically acceptable, as formulated.

Compounds which do not fall into this category may be employed in the synthesis of compounds which do, for example.

The compounds of the present invention may be used according to well known methods of using bisphosphonates and derivatives thereof, e.g. for use in treating conditions characterized by bone loss (e.g., post-menopausal osteoporosis, ovariectomy patients, senile osteoporosis, patients undergoing long-term treatment of corticosteroids, side effects from glucocorticoid or steroid treatment, patients suffering from Cushings's syndrome, gonadal dysgenesis, periarticular erosions in rheumatoid arthritis, osteoarthritis, Paget's disease, osteohalisteresis, osteomalacia, hypercalcaemia of malignancy, osteopenia due to bone metastases, periodontal disease, and hyperparathyroidism). The compounds may also be used prophylactically to prevent the conditions enumerated above.

Useful pharmaceutically acceptable salts include acid addition salts, e.g., salts with inorganic acids such as HCl, HBr, sulfuric, sodium hydrogen sulfate, phosphoric acid, sodium dihydrogen phosphate, and disodium hydrogen phosphate, as well as salts with organic acids such as formic, acetic, benzoic, carbonic and the like. Where the compound is substituted by a carboxy group, pharmaceutically acceptable salts may be obtained with an inorganic base such as an alkali or alkaline earth metal hydroxide (e.g. LiOH, NaOH, KOH, or Ca(OH)₂) or an organic base such as choline hydroxide, spermidine, spermine, glucamine, triethylamine and the like.

The invention further provides glycosides or orthoester glycosides of the bisphosphonate compounds described in U.S. Pat. No. 5,409,91 1. Included in such compounds are prostaglandin derivatives having the Formulae (III) and (IV):

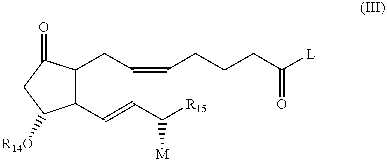

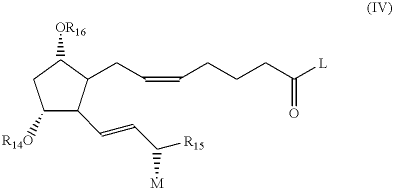

wherein R₁₅ is hydrogen or alkyl;

R₁₄ and R₁₆, which may be the same or different, are hydrogen, a glycoside or orthoester glycoside as described herein, tetrahydropyran (THP) or —Si(CH₃)₂t-Bu;

M is:

(1)—OH or alkoxy;

(2)

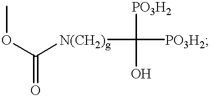

(3)

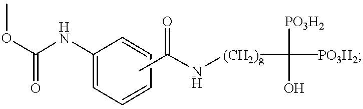

(4)

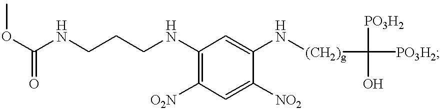

(5)

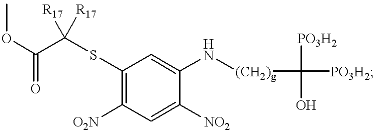

(6)

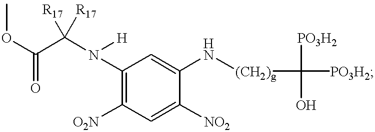

(7)

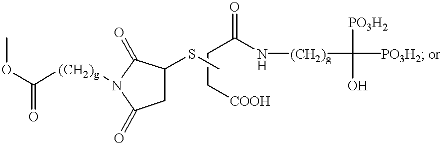

(8)

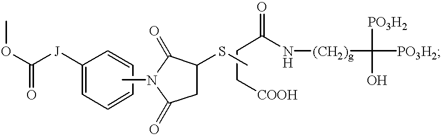

wherein each g, which may be the same or different, is an integer between 1 and 10 and each R₁₇, which may be the same or different, is independently hydrogen, alkyl, aryl or benzyl; and

J is —NH—, —C(R₁₅)₂, or absent;

L is:

(1) —OR₁₈, wherein R₁₈ is alkyl;

(2)

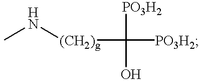

(3)

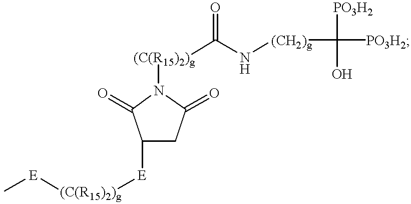

(4)

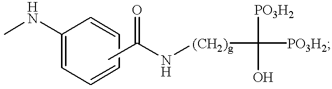

(5)

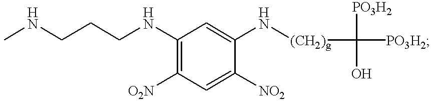

(6)

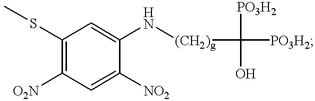

(7)

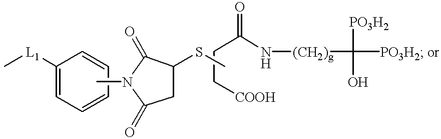

(8)

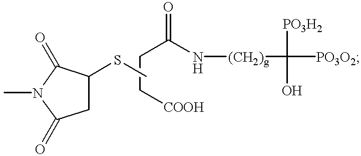

wherein g is defined as above;

E is —NR₁₅—, —O—, or —S—; and

L₁ is —NH—, —C(R₁₅)₂—, or absent;

with the proviso that at least one hydroxy group on the compounds of Formulae (III) and (IV) is substituted by a glycoside or orthoester glycoside as described herein; and

pharmaceutically acceptable salts or esters thereof.

The invention also provides the glycosides and orthoester glycosides of the hydroxy containing bisphosphonate compounds described in U.S. Pat. No. 5,668,120. Included in such compounds are compounds having the Formula (V):

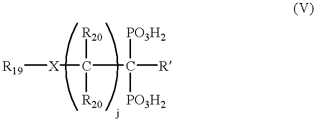

wherein R′ is hydrogen, —NH₂, —OH or Cl;

j is an integer from 0 to 7;

each R₂₀, which may be the same or different, is hydrogen, substituted or unsubstituted, saturated or unsaturated alkyl having from 1 to about 4 carbon atoms;

X′ is —NH—, quaternary amine, oxygen, sulfur, or a single bond; and

R₁₉ is a substituted carbocycle or substituted heterocycle; and pharmaceutically acceptable salts or esters thereof;

with the proviso that at least one hydroxy group on R₁₉ is substituted by a glycoside or orthoester glycoside as described herein.

The invention also provides the glycosides and orthoester glycosides of the hydroxy containing bisphosphonate compounds described in U.S. Pat. No. 5,602,115. Included in such compounds are compounds having the Formula (VI):

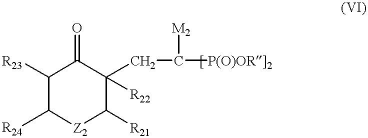

wherein M₂ is hydrogen, Cl, or methyl;

each R″, which may be the same or different, is hydrogen, alkyl, benzyl, phenyl optionally substituted with 1 to 5 substituents selected from nitro, fluoro, chloro, alkyl or where both —OR″ on the same P are taken together with —CH₂CH₂—, —CH₂CH₂CH₂— or —CH₂C(CH₃)₂CH₂— to form a heterocyclic ring containing one —P—, two —O— and two or three carbon atoms;

R₂₁ is alkyl, phenyl optionally substituted with 1 to 5 substituents selected from halo, —NO₂, —CN, —CF₃, alkyl, cycloalkyl, —OH, alkoxy, phenoxy, alkylthio or —NC(O)R₂₅;

R₂₅ is alkyl, naphthalene optionally substituted with 1 or 2 phenyl; or naphthalene optionally substituted with 1 to 7 substituents selected from halo, —NO₂, —CN, —CF₃, alkyl, cycloalkyl, —OH, alkoxy, phenoxy, alkylthio or —N(CH₃)₂;

R₂₂, R₂₃ and R₂₄, which may be the same or different, are hydrogen, alkyl, phenyl optionally substituted with 1 to 5 substituents selected from halo, —NO₂, —CN, —CF₃, alkyl, cycloalkyl, —OH, alkoxy, phenoxy, alkylthio, —NC(O)R₂₅; naphthalene optionally substituted with 1 or 2 phenyl; or naphthalene optionally substituted with 1 to 7 substituents selected from halo, —NO₂, —CN, —CF₃, alkyl, cycloalkyl, —OH, alkoxy, phenoxy, alkylthio or —N(CH3)2;

alternatively R₂₃ and R₂₄ taken together with the atoms to which they are attached form an aryl ring;

Z₂ is —O—, —(CH₂)ₖ—, —S(O)ₖ—, —NZ₃—, —NS(O)₂Z₄—, —NC(O)Z₅;

k is 0, 1 or 2;

Z₃ is hydrogen, alkyl, cycloalkyl, phenyl, 2-pyridinyl, 3-pyridinyl or 4-pyridinyl;

Z₄ is alkyl or phenyl optionally substituted with 1 to 5 substituents selected from halo, —NO₂, —CN, —CF₃, alkyl, cycloalkyl, —OH, alkoxy, phenoxy, or alkylthio;

Z₅, is alkyl; cycloalkyl; 2-, 3-, or 4-pyridinyl; naphthalene optionally substituted with 1 or 2 phenyl; naphthalene optionally substituted with 1 to 7 substituents chosen from halo, —NO₂, —CN, —CF₃, alkyl, cycloalkyl, —OH, alkoxy, phenoxy, alkylthio or —N(CH₃)₂; and

pharmaceutically acceptable salts or esters thereof;

with the proviso that there is at least one hydroxy group on the compound of Formula (VI) that is substituted by a glycoside or orthoester glycoside as described herein.

The invention also provides the glycosides and orthoester glycosides of the hydroxy containing bisphosphonate compounds described in U.S. Pat. No. 5,635,495. Included in such compounds are compounds having the Formula (VII):

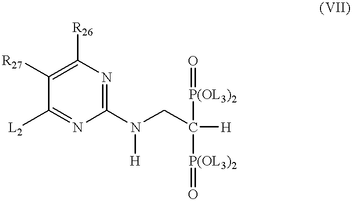

wherein R₂₆ is hydrogen, alkyl, cycloalkyl, or phenyl optionally substituted with 1 or 2 phenyl or 1 to 5 halo, nitro, cyano, —CF₃, alkyl, cycloalkyl, —OH, —SH, —NH₂, alkoxy or alkylthio groups;

R₂₇ is hydrogen, phenyl, halo, alkyl, cycloalkyl, —NH₂, alkoxy, or alkylthio;

each L₃, which may be the same or different, is hydrogen, alkyl, cycloalkyl, benzyl or where both L₃ on a single phosphorus are —CH₂CH₂—, —CH₂CH₂CH₂— or —CH₂C(CH₃)₂CH₂—, whereby a heterocyclic ring is formed;

L₂ is —OR″′, —SR″′, morpholinyl, piperazinyl, piperidinyl, imidazolidinyl, pyrazolidinyl, isoxazolyl, pyrrolyl, imidazolyl, pyrazolyl, pyridyl, pyrazinyl, pyrimidinyl, or pyridazinyl;

wherein R′″ is phenyl optionally substituted with 1 or 2 phenyls, or 1 through 5 halo, —NO₂, —CN, —CF₃, alkyl, cycloalkyl, —OH, —SH, —NH₂, alkoxy or alkylthio groups;

in the case where R″′ is one of the listed heterocyclic rings, the attachment point is at an available ring nitrogen; and

pharmaceutically acceptable salts or esters thereof;

with the proviso that there is at least one hydroxy or amino group on the compound of Formula (VII) that is substituted by a glycoside or orthoester glycoside as described herein.

The invention also provides the glycosides and orthoester glycosides of the bisphosphonate compounds described in U.S. Pat. No. 5,763,61 1. Included in such compounds are compounds of the Formula (VIII):

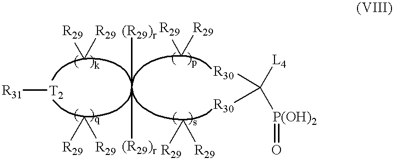

wherein L₄ is —PO₃H₂ or —P(O)OHL₅; wherein L₅ is substituted or unsubstituted alkyl;

T₂ and R₃₀, which may be the same or different, are absent or are O, S or N;

k and q are integers from 0 to 5 and k+q equals 0 to 5;

p and s are integers from 0 to 3 and p+s is 0 to 3;

r is an integer from 0 to 2 and when T₂is absent and k+q is 0, then r is 2;

each R₂₉, which may be the same or different, is absent or independently selected from —SR₃₂, —R₃₃SR₃₂, hydrogen, unsubstituted or substituted alkyl, unsubstituted or substituted aryl, hydroxy, amido, alkoxy, —CO₂R₃₄, —N(R₃₄)₂, —N(R₃₄)CO, —OR₃₄, —C(O)N(R₃₄)₂ or substituted or unsubstituted benzyl;

with the proviso that when one R₂₉ is absent, the adjacent R₂₉ must also be absent thereby indicating an unsaturation;

R₃₁ is absent, —SR₃₂, —R₃₃SR₃₂, hydrogen, unsubstituted or substituted alkyl, unsubstituted or unsubstituted aryl, hydroxy, amido, —CO₂R₃₄, —O₂CR₃₄, —NR₃₄C(O)R₃₄, —OR₃₄, —N(R₃₄)₂, —C(O)N(R₃₄)₂, substituted or unsubstituted benzyl, nitro, or combinations thereof;

R₃₂ is hydrogen, —C(O)R₃₅, —C(S)R₃₅, —C(O)N(R₃₅)₂, —CSN(R₃₅)₂, —C(O)OR₃₅ or —C(S)OR₃₅;

R₃₃ is substituted or unsubstituted alkyl, provided that at least one of R₂₉, R₃₁, and R₃₄ is —SR₃₂ or —R₃₃SR₃₂;

R₃₄ is hydrogen, substituted or unsubstituted alkyl or —R₃₃SR₃₂;

R₃₅ is hydrogen, or substituted or unsubstituted alkyl; and

pharmaceutically acceptable salts or esters thereof;

with the proviso that there is at least one hydroxy group on the compound of Formula (VIII) that is substituted by a glycoside or orthoester glycoside as described herein.

The invention further provides the glycosides and orthoester glycosides of the bisphosphonate compounds described in U.S. Pat. No. 5,183,815. Included in such compounds are steroid derivatives of the Formula (IX):

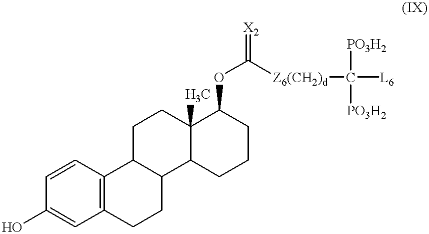

wherein L₆ is hydrogen or —OH;

X₂ is O or S;

Z₆ is —NH—, —O—, —NR₃₆—;

R₃₆ is hydrogen or alkyl;

d is an integer from 1-4; and

pharmaceutically acceptable salts or esters thereof;

with the proviso that the hydroxy group at carbon-3 is substituted by a glycoside or orthoester glycoside as described herein.

The invention also provides the glycosides or orthoester glycosides of the bisphosphonate compounds described in U.S. Pat. No. 5,428,181. Included in such compounds are compounds of the general Formula (X):

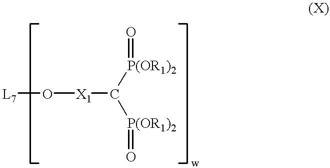

wherein L₇ denotes a residue of a compound having an oestrogenic activity substituted by a glycosidoxy or orthoester glycosidoxy group;

each R₁, which may be the same or different, denotes hydrogen or a C₁-C₆ alkyl group;

X₁ denotes a single bond, a C₁-C₁₀ alkylene group or a group of the formula:

[chemical structure]

wherein R₃₇ denotes hydrogen or a C₁-C₅ alkyl group;

V denotes a nitro group or a halogen;

t is an integer of 3 to 12;

u is an integer of 1 to 5;

v is an integer of 0 to 5;

w is an integer of 1 to 3; and

pharmaceutically acceptable salts and esters thereof.

Example compounds possessing oestrogenic activity include oestradiol, oestriol, genistein, daidzein, coumestrol, hexestrol and stilbestrol.

A group of preferred compounds of the invention has the Formula (XI):

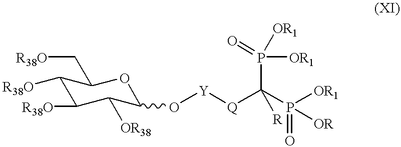

wherein R₃₈ is selected from the group consisting of hydrogen, acetyl, benzoyl, nicotinoyl, benzyl, methyl and phenyl; and each R, R₁, Y and Q is as defined; and pharmaceutically acceptable salts and esters thereof.

Preferably, each R₁ in compounds of the Formula (XI) is selected from the group consisting of hydrogen, alkyl, benzyl and phenyl.

Preferred meanings of some of the terms used herein are as follows:

“alkyl”—straight- or branched-chain hydrocarbons having from 1 to 10 carbon atoms and more preferably 1 to 8 carbon atoms;

“substituted alkyl”—an alkyl group, preferably containing from 1 to 10 carbon atoms, having from 1 to 5 substituents selected from the group consisting of alkoxy, substituted alkoxy, acyl, amino, aryl, substituted aryl, aryloxy, substituted aryloxy, cyano, halogen, hydroxyl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxyl-heteroaryl, carboxyl-substituted heteroaryl, carboxylheterocyclic, carboxyl substituted heterocyclic, cycloalkyl, substituted cycloalkyl, -SH, thioalkyl, substituted thioalkyl, thioaryl, substituted-thioaryl, thiocycloalkyl, substituted-thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy or substituted cycloalkoxy;

“allyl”—the group “CH₂═CH—CH₂—”;

“alkoxy”the group “alkyl O—”which includes, by way of example, methoxy, ethoxy, n-propoxy, i-propoxy, n-butyl, t-butyl and the like;

“substituted alkoxy”—the group “substituted alkyl-O—”;

“cycloalkoxy”—the group “cycloalkyl-O—”;

“substituted cycloalkoxy”—the group “substituted cycloalkyl-O—”;

“thioalkyl”or “alkylthio”—the group “alkyl-S—”;

“substituted thioalkyl”—the group “substituted alkyl-S—”;

“thiocycloalkyl”—the group “cycloalkyl-S—”;

“substituted thiocycloalkyl”—the group “substituted cycloalkyl-S—”;

“thioaryl”—the group “aryl-S—”;

“thioheteroaryl”—the group “heteroaryl-S—”;

“substituted thioheteroaryl”—the group “substituted heteroaryl-S—”;

“thioheterocyclic”—the group “heterocyclic-S—”;

“substituted thioheterocyclic”—the group “substituted heterocyclic-S—”;

“substituted thioaryl”the group “substituted aryl-S—”;

“amino”the group —NH₂;

“substituted amino”—the group —NRR, where each R group is independently selected from the group consisting of hydrogen, alkyl, substituted alkyl, cycloalkyl, substituted cycloalkyl, aryl, substituted aryl, heteroaryl, substituted heteroaryl, heterocyclic or substituted heterocyclic, provided that not both are hydrogen; or the R groups can be joined together with the nitrogen to form a heterocyclic ring;

“acyl”-alkanoyl groups, e.g. HC(O)—, alkyl-C(O)—, substituted-alkyl-C(O)—, cycloalkyl-C(O)—, substituted-cycloalkyl-C(O)—, aryl-C(O)—, substituted-aryl-C(O)—, heteroaryl-C(O)—, substituted-heteroaryl-C(O)—, heterocyclic-C(O)— or substituted heterocyclic-C(O)—, wherein substituted alkyl, cycloalkyl, aryl and heteroaryl are defined and exemplified herein;

“aryl”—an unsaturated aromatic carbocyclic group of 6 to 14 carbon atoms having a single ring (e.g., phenyl) or multiple condensed rings (e.g., naphthyl or anthryl);

“aryloxy”—the group “aryl-O—”;

“substituted aryl”—aryl group substituted with 1 to 3 substituents selected from hydroxy, acyl, alkyl, substituted alkyl, alkoxy, substituted alkoxy, amino, aryl, substituted aryl, carboxyl, carboxylalkyl, carboxyl-substituted alkyl, carboxyl-cycloalkyl, carboxyl-substituted cycloalkyl, carboxylaryl, carboxyl-substituted aryl, carboxyl-heteroaryl, carboxyl-substituted heteroaryl, carboxylheterocyclic, carboxyl substituted heterocyclic, cycloalkyl, substituted cycloalkyl, —SH, thioalkyl, substituted thioalkyl, thioaryl, substituted-thioaryl, thiocycloalkyl, substituted-thiocycloalkyl, thioheteroaryl, substituted thioheteroaryl, thioheterocyclic, substituted thioheterocyclic, halogen, nitro, heteroaryl, substituted heteroaryl, heterocyclic, substituted heterocyclic, cycloalkoxy or substituted cycloalkoxy;

“cycloalkyl”—cyclic alkyl groups containing between 3 and 8 carbon atoms having a single cyclic ring including, by way of example, cyclopropyl, cyclobutyl, cyclopentyl, cyclooctyl and the like;

“substituted cycloalkyl”—a cycloalkyl group containing between 3 and 8 carbon atoms, having from 1 to 5 substituents selected from the group consisting of oxo (═O), thioxo (═S), alkoxy, substituted alkoxy, acyl, amino, aryl, substituted aryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxyl-heteroaryl, carboxyl-substituted heteroaryl, carboxylheterocyclic, carboxyl substituted heterocyclic, cycloalkyl, substituted cycloalkyl, —SH, thioalkyl, substituted thioalkyl, thioaryl, substituted-thioaryl, thiocycloalkyl, substituted-thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy or substituted cycloalkoxy;

“halogen” or “halo”—fluoro, chloro, bromo, iodo;

“heteroaryl”—an aromatic carbocyclic group containing from 2 to 10 carbon atoms and 1 to 4 heteroatoms within the ring. Such heteroaryl groups can have a single ring (e.g., pyridyl or furyl) or multiple condensed rings (e.g., indolizinyl or benzothienyl);

“substituted heteroaryl”—groups which are substituted with 1 to 3 substituents consisting of hydroxy, acyl, alkyl, substituted alkyl, alkoxy, substituted alkoxy, amino, aryl, substituted aryl, cycloalkoxy, substituted cycloalkoxy, carboxyl, carboxylalkyl, carboxyl-substituted alkyl, carboxyl-cycloalkyl, carboxyl-substituted cycloalkyl, carboxylaryl, carboxyl-substituted aryl, carboxyl-heteroaryl, carboxyl-substituted heteroaryl, carboxylheterocyclic, carboxyl substituted heterocyclic, cycloalkyl, substituted cycloalkyl, —SH, thioalkyl, substituted thioalkyl, thioaryl, substituted-thioaryl, thiocycloalkyl, substituted-thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkyl, substituted cycloalkyl, halo, nitro, heteroaryl or substituted heteroaryl;

“heterocycle”—a saturated or unsaturated group having a single ring or multiple condensed rings, containing 1 to 10 carbon atoms and 1 to 4 heteroatoms within the ring, wherein in fused ring systems, one or more of the rings can be aryl or heteroaryl;

“substituted heterocycle”—heterocyclic groups which are substituted with 1 to 3 substituents selected from the group consisting of oxo (═O), thioxo (═S), alkoxy, substituted alkoxy, acyl, amino, aryl, substituted aryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxyl-heteroaryl, carboxyl-substituted heteroaryl, carboxylheterocyclic, carboxyl substituted heterocyclic, cycloalkyl, substituted cycloalkyl, —SH, thioalkyl, substituted thioalkyl, thioaryl, substituted-thioaryl, thiocycloalkyl, substituted-thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy or substituted cycloalkoxy;

“heteroatoms”—oxygen, nitrogen and sulfur;

“benzyl”—the group “—CH₂-phenyl”;

“substituted benzyl”—the group “—CH₂-substituted-phenyl”;

“aralkyl”—the group “-alkylaryl”;

“leaving group” includes, but is not limited to, chloride, bromide, iodide, tosylate, mesylate and triflate;

“pharmaceutically acceptable salts” includes, but is not limited to, alkali metals (e.g., sodium and potassium), alkaline earth metals (e.g., calcium and magnesium), non-toxic heavy metals (e.g., stannous and indium) or ammonium and low molecular weight substituted ammonium (e.g., mono-, di- and triethanolamine) salts, preferred salts being the sodium, potassium, and ammonium salts; and

“pharmaceutically acceptable esters”—unsubstituted and substituted alkyl, aryl or phosphoryl esters, non-limiting examples including, i-propyl, t-butyl, 2-chloroethyl, 2,2,2-trichloroethyl, 2,2,2-trifluoroethyl, p-toluenesulphonylethyl, glycyl, sarcosyl, benzyl, phenyl, 1,2-hexanoylglyceryl, p-nitrophenyl, 2,2-dimethyl-1,3-dioxolene-4-2,2 dimethyl-1,3-dioxolene-4-methyl, i-pentenyl, o-carbomethoxyphenyl, pivaloyloxymethylsalicylyl, diethylamidophosphoryl, pivaloyloxymethyl, acyloxymethyl, propionyloxymethyl, i-butyryloxymethyl, dodecyl, octadecyl, and i-propyloxymethyl.

Most preferred compounds include, but are not limited to, 4-O-glycofuranosides, 4-O-glycopyranosides, straight chained or branched 4-O-oligoglycosides and orthoester glycosides of 4-hydroxybutane-1,1-(tetra-O-isopropyl)bisphosphonate, 4-hydroxybutane-1,1-bisphosphonic acid and 4-N-glycopyranosides and 4-N-glycofuranosides of alendronate.

Preferred glycosidic units have free hydroxy groups, or hydroxy groups protected with aryl (e.g. phenyl), —CH₂-aryl (e.g. benzyl), aroyl (e.g. benzoyl), heteroaroyl (e.g. nicotinoyl), alkyl (e.g. methyl), or alkanoyl (e.g. acetate) groups.

The water soluble glycosidic derivatives of the aforementioned bisphosphonates may be obtained according to the general methods disclosed by Holick in U.S. Pat. No. 4,410,515, the contents of which are fully incorporated by reference herein.

The compounds of the present invention can be administered in any appropriate pharmaceutically acceptable carrier for oral administration, since the glycosides and orthoester glycosides of bisphosphonate derivatives are biologically active upon oral administration. The compounds of the invention may also be administered in any appropriate pharmaceutical carrier for parenteral, intramuscular or topical administration. They can be administered by any means that treat and/or prevent conditions treatable with a bisphosphonate (e.g., hypercalcaemia of malignancy, Paget's disease, osteoporosis, metastatic cancer in bone and soft tissue and periodontal disease).

The dosage administered will depend on the age, health and weight of the recipient, kind of concurrent treatment, if any, frequency of treatment and the nature of the effect desired. An exemplary systemic daily dosage is about 0.1 mg to about 500 mg. Normally, from about 10 mg to 100 mg daily of the glycoside or orthoester glycoside, in one or more dosages per day, is effective to obtain the desired results. One of ordinary skill in the art can determine the optimal dosages and concentrations of other active glycosides of bisphosphonate derivatives with only routine experimentation.

The compounds can be employed in dosage forms such as tablets and capsules for oral administration, as well as sterile liquid for formulations such as solutions or suspensions for parenteral use. A lipid vehicle can be used in parenteral administration. The compounds may also be administered via topical patches, ointments, gels or other transdermal applications. In such compositions, the active ingredient will ordinarily be present in an amount of at least 0.001% by weight based on the total weight of the composition, and not more than 50% by weight. An inert pharmaceutically acceptable carrier is preferable such as 95% ethanol, vegetable oils, propylene glycols, saline buffers, sesame oil, etc. Reference is made to Remington's Pharmaceutical Sciences, 18^{th} Edition, Gennaro, et al. (eds.), 1990, for methods of preparing pharmaceutical compositions.

For oral administration, the compounds of the invention can be administered in any appropriate pharmaceutically acceptable carrier. The compounds of the invention may also be administered in any appropriate pharmaceutical carrier for parenteral, intramuscular, transdermal, intravenous, intranasal or inhalation administration. They can be administered by any means that achieve their intended purpose.

The compounds can be employed in dosage forms such as tablets, capsules or powder packets, or liquid solutions, suspensions or elixirs for oral administration, as well as sterile liquid for formulations such as solutions or suspensions for parenteral use. A lipid vehicle can be used in parenteral administration. The compounds may also be administered via topical patches, ointments, gels, liposomes or other transdermal applications. In such compositions, the active ingredient will ordinarily be present in an amount of at least 0.01% by weight based on the total weight of the composition, and not more than 90% by weight. An inert pharmaceutically acceptable carrier is preferable such as 95% ethanol, vegetable oils, propylene glycols, saline buffers, sesame oil, etc. Reference is made to Remington's Pharmaceutical Sciences, 18^{th} Edition, Gennaro et al. (eds.), 1990, for methods of preparing pharmaceutical compositions.

Topical formulations for transdermal, intranasal or inhalation administration may be prepared according to methods well known in the art. For topical administration, the compounds may be applied in any of the conventional pharmaceutical forms. For example, the compounds may be administered as part of a cream, lotion, aerosol, ointment, powder, liposomes or drops. Ointments and creams may, for example, be formulated with an aqueous or oily base with the addition of suitable thickening and/or gelling agents. Such bases may include water and/or an oil such as liquid paraffin or a vegetable oil such as peanut oil or castor oil. Thickening agents which may be used include soft paraffin, aluminum stearate, cetostearyl alcohol, polyethylene glycols, wool-fat, hydrogenated lanolin, beeswax and the like.

Lotions may be formulated with an aqueous or oily base and will in general also include one or more of a stabilizing agent, thickening agent, dispersing agent, suspending agent, thickening agent, coloring agent, perfume and the like.

Powders may comprise any suitable powder base including talc, lactose, starch and the like. Drops may comprise an aqueous or non-aqueous base together with one or more dispersing agents, suspending agents, solubilizing agents and the like.

Preferred liposomes are NOVASOMES sold by IGI, Inc. (Buena, N.J.) and described in U.S. Pat. No. 5,260,065.

The compositions may further comprise one or more preservatives including bacteriostatic agents including methyl hydroxybenzoate, propyl hydroxybenzoate, chlorocresol, benzalkonium chloride and the like.

The topical compositions comprise from about 0.0001% to 10% by weight, preferably, 0.001 to 0.5% by weight, more preferably, 0.01 to 0.25% by weight.

The compounds may be administered either individually or as a mixture containing non-glycosylated, monoglycosylated, and/or bisglycosylated compounds, or a mixture of the corresponding orthoester glycosides.

The compounds are preferably provided substantially pure prior to formulation. The phrase “substantially pure” encompasses compounds created by chemical synthesis and/or compounds substantially free of chemicals which may accompany the compounds in the natural state, as evidenced by thin layer chromatography (TLC) or high performance liquid chromatography (HPLC).

Animals which may be treated according to the methods of the present invention include all animals which may benefit therefrom. Included in such animals are humans, although the invention is not so limited.

The invention further provides a method of preparing a compound of Formula (XI), as illustrated in Scheme I below:

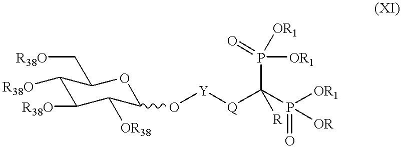

wherein R₁, R, Y and Q are as defined, Y and Q together preferably representing a propylidene group and R preferably being a hydrogen, and R₃₈ is selected from hydrogen, acetyl, benzoyl, nicotinoyl, benzyl, methyl and phenyl;

which method comprises:

(a) reacting a glycoside having the Formula (XII):

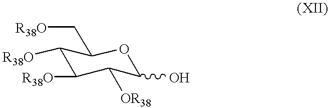

wherein R₃₈ is other than hydrogen;

with a group of formula Hal—Y—Q—X₃, wherein Hal and X₃ individually represent halogen, and is preferably a 1,3-dihaloalkane, in the presence of a strong base in an aprotic solvent to give a compound of Formula (XIII):

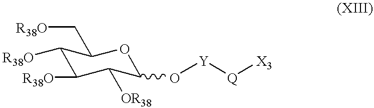

wherein X₃ is as defined; and

(b) reacting the compound of Formula (XIII) with a compound of the Formula (XIV):

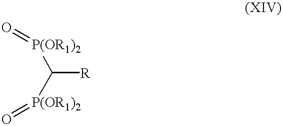

wherein R and R₁ are as defined, in the presence of a strong base in an aprotic solvent to give a compound of Formula (XI), wherein R₁ and R₃₈ are other than hydrogen. The groups R₁ and/or R₃₈ may then be partially or fully removed, preferably by hydrolysis.

The invention further provides a method of preparing a compound of Formula (XV):

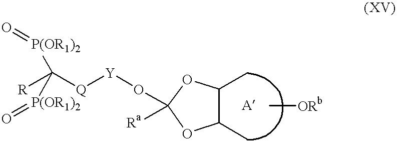

wherein R^{a}, R^{b}, R₁, R, A′, Y and Q are as defined, Y and Q together preferably representing a propylidene group and R preferably being a hydrogen, R₂ preferably being hydrogen;

which method comprises:

(a) reacting a glycoside having the Formula (XVI):

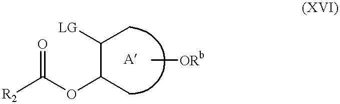

wherein R^{b} has protecting groups on each hydroxyl; and

LG is a leaving group;

with an alcohol of the formula (XVII):

P′O—Q—Y—OH (XVII)

wherein P′ is a protecting group;

in the presence of a non-nucleophilic base in an aprotic solvent to give a compound of Formula (XVIII):

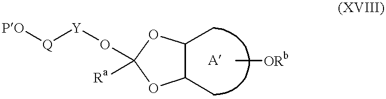

(b) replacing P′ with a leaving group, preferably by removing the protecting group by known methods, such as treatment with fluoride (e.g., tetrabutylammonium fluoride where P′ is a trialkylsilyl or an alkyldiphenylsilyl group). The resulting alcohol may then be converted into a leaving group, LG′, thus giving a compound of the Formula (XIX):

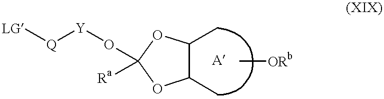

(c) reacting the compound of Formula (XIX) with a compound of the Formula (XIV):

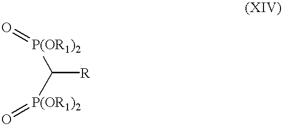

in the presence of a strong base in an aprotic solvent to give the compound of Formula (XV). The groups R₁ and/or the protecting groups on the orthoester glycoside may then be partially or fully removed, preferably by hydrolysis.

Examples of LG and LG′, which may be the same or different, include bromine, chlorine, iodine, tosylate, mesylate, triflate, and the like.

Examples of protecting groups, P′, include trialkylsilyl groups (e.g., TIPS), aryldialkylsilyl groups (e.g., TBDPS) and the like.

Examples of non-nucleophilic bases include collidine, lutidine and the like.

Examples of protecting groups that may be present on the hydroxyl groups of the glycosidic residues of compounds of the Formula (XV), (XVI), (XVIII) and (XIX), include acetyl, benzoyl, nicotinoyl, benzyl, methyl, phenyl and the like.

Examples of strong bases that may be used in the synthesis of compounds of Formula (XI), (XIII) and (XV) include hydrides of sodium, potassium and lithium, anion of dimethylsulphoxide (dimisyl anion), potassium and sodium salts of bistrimethylsilylamide and the like.

The reactions are carried out in an aprotic solvent such as dimethylformamide, THF, toluene, acetonitrile, dimethylsulphoxide and the like.

The reaction temperatures are from about −10 to 25° C. Preferably, the reaction temperature is at about 0° C. The reaction may be carried out for about 1 to 10 hours, or until TLC shows that the reaction is complete.

The thus formed compound of Formula (XI) or (XV) is then isolated and may be purified, for example, on a silica gel column or by HPLC. The compounds of Formula (XI) or (XV) have protecting groups on the glycoside or orthoester glycoside moiety and groups (R₁) on the bisphosphonate moiety that may also be partially or fully removed. R₁ groups on the bisphosphonate moiety may be removed by any known methods including treatment with trimethylsilyl bromide.

When the protecting groups on the glycoside or orthoester glycoside moiety are benzyl groups, then they may be removed by any known methods including treatment with trimethylsilyl bromide or by hydrogenation. When the protecting groups on the glycoside or orthoester glycoside moiety are acetyl, benzoyl or nicotinoyl, then they may be removed by any known methods including treatment with alkali alkoxide in alcohol (e.g., sodium methoxide in methanol). When the protection groups on the glycoside or orthoester glycoside moiety are methyl, then they may be removed by any known methods including treatment with a Lewis acid (e.g., AlCl₃ in combination with (n-Bu)₄NI).

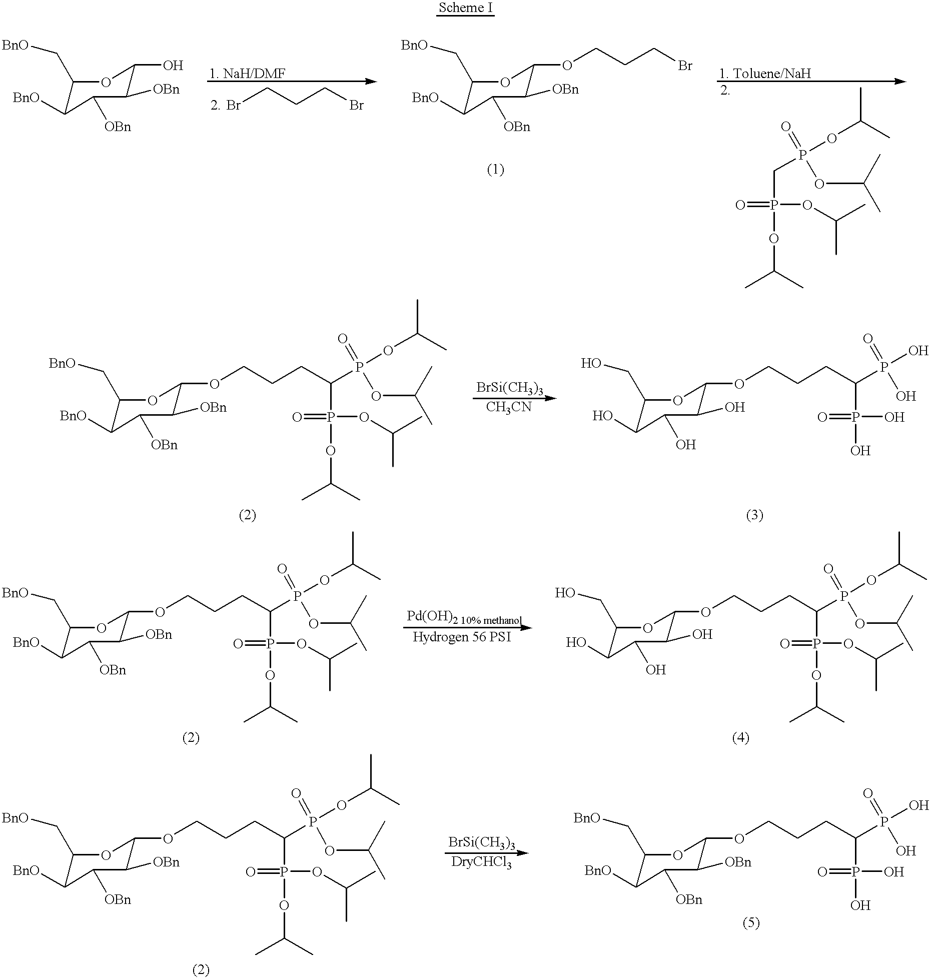

In the above scheme, a synthesis involving L-sugars is illustrated, although it will be appreciated that the same reaction scheme also applies to the corresponding D-sugars.

The present invention further provides a process for the preparation of a compound of formula (XX):

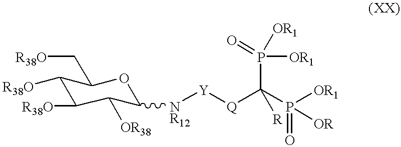

wherein Y, Q, R, R₁, R₁₂ and R₃₈ are as defined, said method comprising dissolving a compound of formula (XXI):

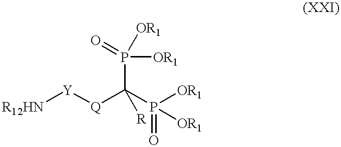

in an aqueous medium, preferably water, together with sufficient of a trialkylamine compound to form a salt thereof;

removing the water;

dissolving the salt in a water miscible, organic solvent, such as methanol; adding a compound of formula (XII):

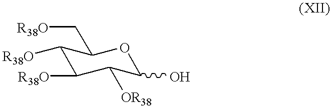

wherein R₃₈ is as defined, and maintaining the resulting mix under such conditions as to form a trialkylamine salt of the compound of formula (XX). The trialkylamine may then be removed by conventional means, such as by running the salt on a cationic exchange resin.

Preferred aqueous media or solvents are distilled or deionized water. It is preferred to use just water, but other, water miscible, organic solvents may also form up to about 50% of the medium.

Any suitable trialkylamine, such as trimethylamine or triethylamine, may be used, although it should be able to readily form a soluble salt of the compound of formula (XXI). In this respect, it should be noted that the compound of formula (XXI) is not necessarily readily soluble in water, but that addition of trialkylamine brings it into solution by converting it into a salt.

The water may be removed by any suitable method, such as evaporation, especially under pressure at an elevated temperature. It is not necessary to completely dry the mix beyond that degree obtainable by evaporation.

Suitable water miscible, organic solvents include methanol, ethanol and tetrahydrofuran. What is necessary is to be able to dissolve both the salt of compound (XXI) and the glycoside compound (XII).

The resulting mix may need to be warmed to drive the glycosidic compound into solution, after which the solution may be maintained, preferably with agitation, such as stirring, at ambient temperature, while the glycosylation reaction proceeds. This may take up to two days, or more.

Having now generally described this invention, the same will be understood by reference to the following examples which are provided herein for purposes of illustration only and are not intended to be limiting unless otherwise specified.

Examples 1 to 3 relate to the synthesis of (4-O-(1′-glycopyranosyl))-4-hydroxybutane-1,1-bisphosphonic acid, tetra-O-isopropyl-(4-O-(1′-glycopyranosyl))-4-hydroxybutane-1,1-bisphosphonate and 4-O-((2′,3′,4′,6′-tetra-O-benzyl)-1′-glyco-pyranosyl))-4-hydroxybutane-1,1-bisphosphonic acid (Scheme I).

### EXAMPLE 1

### A. Synthesis of 3-Bromo-1-O-((2′,3′,4′,6′-tetra-O-benzyl)glucopyranosyl)propanol (1) (as a mixture of α and β anomers).

To a stirred suspension of sodium hydride (60% dispersion in mineral oil, 900 mg, 1.3 equivalents) in dry dimethylformamide (50 ml) at 0° C. under argon atmosphere was added 2,3,4,6-tetra-O-benzyl glucopyranose (9.3 g, 17 mmole). The mixture was stirred until the evolution of hydrogen ceased. A solution of 1,3-dibromopropane (5.18 g, 1.5 equivalents) in dimethylformamide (50 ml) was added in a single portion. The mixture was warmed to room temperature and stirred for an additional 4 hours. After pouring the reaction mixture into ice cold water (350 ml), the product was extracted with ether (3×150 ml). The combined ether extracts were washed once with water and dried over magnesium sulfate. After evaporation of the solvent and silica gel chromatography with 10% ethyl acetate-hexane, the title product (5.8 g) was obtained as a mixture of anomers.

¹H NMR (CDCl₃)

δ7.3-7.0 (m, Ar—H, 20H),

δ5.3-3.3 (m, complex multiplets, 19H),

δ2.2-2.0 (m, CH₂).

### B. Synthesis of Tetra-O-isopropyl-(4-O-(2′,3′,4′,6′-tetra-O-benzyl)glucopyranosyl)-4-hydroxybutane-1,1-bisphosphonate (2) (as a mixture of α and β anomers).

To a stirred suspension of sodium hydride (200 mg, 60% dispersion in mineral oil, 1.5 equivalents) in dry toluene (50 ml) was added tetraisopropyl methylenebisphosphonate (1.6 g, 1.4 equivalents) at 0° C. under argon. After evolution of hydrogen had ceased, a solution of 3-bromo-1-O-((2′,3′,4′,6′-tetra-O-benzyl)glucopyranosyl)propanol (1) (2.2 g, 3.3 mmol) in dry toluene (10 ml) was added in a single portion. The mixture was warmed slowly to 80° C. The mixture was stirred at 80° C. for a period of 8 hours. The mixture was cooled, poured into water (150 ml) and extracted with ether (3×100 ml). The combined organic extracts were washed with water (2×100 ml) and dried over magnesium sulfate. Following evaporation and silica gel column chromatography using ethyl acetate-hexane (8:1) the desired title compound (2.5 g) was obtained.

¹H NMR (CDCl₃)

δ7.3-7.0 (m, ArH, 20H),

δ4.9-3.2 (m, 17H),

δ2.2-1.8 (m, 9H),

δ1.25 (d, CH₃, 24H);

MS m/z 925.5 (M+), expected m/z 925.

### C. Synthesis of 4-O-(1′-glucopyranosyl)-4-hydroxybutane-1,1-bisphosphonic acid (3) (as a mixture of α and β anomers)

A solution of tetra-O-isopropyl-(4-0-(2′,3′,4′,6′-tetra-O-benzyl)glucopyranosyl)-4-hydroxybutane-1,1-bisphosphonate (2) (1 g) in dry acetonitrile (10 ml) was cooled to 0° C. under argon. Trimethylsilyl bromide (5 ml) was added in a single portion. The mixture was allowed to warm to room temperature and was stirred for 36 hours. Methanol (10 ml) was added and the mixture was concentrated under reduced pressure (0.2 mm Hg). The residue was lyophilized to remove benzyl bromide. The reddish residue was dissolved in deionized water (5 ml) and passed through an ion exchange column (Dowex-110-OH, 25 g dry resin). The resin was washed with water (40 ml) and the water wash was discarded. The column was washed with aqueous 2% hydrochloric acid (100 ml). The acidic fraction was lyophilized and the product (428mg) was obtained as an unrecrystallizable oil.

¹H NMR (CD₃OD)

δ4.0-3.2 (m, 10H),

δ2.5-1.6 (m, complex due to phosphorus-hydrogen coupling, 4H);

³¹P NMR (CD₃OD, external reference standard of phosphoric acid (85%)) shows a singlet at δ27.03.

### EXAMPLE 2

### Synthesis of Tetra-O-isopropyl-(4-O-(1′-glucopyranosyl))-4-hydroxybutane-1,1-bisphosphonate (4) (as a mixture of α and β anomers)

A solution of tetra-O-isopropyl-(4-O-(2′,3′,4′,6′-tetra-O-benzyl)glucopyranosyl)-4-hydroxybutane-1,1-bisphosphonate (2) (1.8 g) in methanol (125 ml) was mixed with palladium hydroxide (600 mg; 10%, wet de Gussa type). The mixture was shaken under a hydrogen atmosphere at 55 psi in a Parr hydrogenator. After 12 hours, the catalyst was filtered off and the solvent was evaporated under reduced pressure to afford a viscous paste (1.1 g). The product was quite pure by spectral data. Attempts to recrystallise the product failed.

¹H NMR (CD₃OD)

δ3.2-4.0 (sugar-H and CHP₂, 10H),

δ1.6-2.5 (m, 8H),

δ1.1-1.2 (overlapping doublets, CH₃, 24H);

³¹P NMR (CDCl₃, external reference standard of phosphoric acid (85%)) 22.5 (s);

MS (in aqueous 1% ammonia solution) m/z 564.15 (M+), expected m/z 564.49.

### EXAMPLE 3

### Synthesis of 4-O-((2′,3′,4′,6′-tetra-O-benzyl)glucopyranosyl)-4-hydroxybutane-1,1-bisphosphonic acid (5) (as a mixture of α and β anomers)

A solution of tetra-O-isopropyl-(4-O-(2′,3′,4′,6′-tetra-O-benzyl)glucopyranosyl)-4-hydroxybutane-1,1-bisphosphonate (2) (1 g) in dry chloroform (10 ml) was cooled to 0° C. under argon. Trimethylsilyl bromide (2 ml) was added and the solution was stirred for 24 hours at room temperature. Excess trimethylsilyl bromide was removed under reduced pressure and methanol (10 ml) was added. The product was filtered through silica gel and eluted with 1:1 methanol-ethyl acetate. The product (450 mg) was obtained as brownish crystals. The product was characterized by spectral means. The composition of the product is homogenous by TLC (50% methanol-ethyl acetate).

¹H NMR (CD₃OD):

δ6.8-7.2 (m, Ar—H, 20H),

δ3.2-4.8 (m, Ar—CH₂ and sugar-H, 17H),

δ1.5-2.2 (m, tether CH₂, 4H).

³¹P NMR (CD₃OD, external reference standard of Phosphoric acid (85%)) shows a single peak at δ27.0;

MS m/z 755.3 (M+), expected m/z 755.

### EXAMPLE 4

### Synthesis of 1-((4-hydroxyphenyl)amino)ethylidene-1,1-diphosphonic acid & 1-{((4-O-glucopyranosyloxy)phenyl)amino}ethylidene-1,1-diphosphonic acid

Aminobisphosphonic acids bearing 4-hydroxyphenyl and 4-O-glucopyranosyl-oxyphenyl substituents were synthesized utilizing Beckmann rearrangement and in situ trapping of the cations with triethyl phosphite. The reaction scheme is illustrated below.

### 1. Synthesis of 4-methoxyacetophenone oxime

To a solution of aqueous methanol (500 ml methanol and 200 ml water) was added hydroxylamine hydrochloride (32 g) and 4-methoxyacetophenone (50 g). This was warmed to reflux. Potassium hydroxide (20 g) was dissolved in water (40 ml) and introduced into the hot mixture slowly. The pH of the solution was maintained between 6 and 7. The reflux was continued for 4 hours and TLC analysis showed the reaction to be essentially complete. The product was isolated by pouring into water (1 liter) and extracting into dichloromethane (3×200 mol). Evaporation of the solvent afforded the oxime (42 g) as a solid.

### 2. Synthesis of tetraethyl 1-((4-methoxyphenyl)amino)ethylidene-1,1-diphosphonate

A mixture of 4-methoxyacetophenone oxime (4.78 g 30 mMol), triethyl phosphite (11.62 g; 70 mMol) and dry dichloromethane (60 ml) was cooled to 4° C. and stirred efficiently. Phosphorus oxychloride (9.21 g; 60 mMol) was introduced slowly over a period of 10 minutes. The mixture was allowed to warm to room temperature and stirred for an additional period of 14 hours. The reaction mixture was quenched by adding into crushed ice (100 g) containing ammonium hydroxide (50 ml). The mixture was allowed to warm to room temperature and stirred for 2 hours. The mixture was extracted with chloroform (2×120 ml). The combined organic portion was washed once with water (100 ml) and dried over magnesium sulfate. Evaporation of the solvent and subsequent purification over silica gel (using chloroform and methanol mixture) afforded the title compound (3.62 g).

a. Proton NMR spectrum in CDCl₃:

δ6.7(doublet, Ar—H, 2 H) and δ6.9 (doublet, Ar—H, 2 H);

δ4.2 (broad multiplets, O—CH₂—CH₃, 8H);

δ3.75 (singlet, OCH₃, 3H),

δ1.55 (triplet, CH₃, 3H) and

δ1.3 (overlapping triplets, O—CH₂—CH₃, 12H).

b. UV spectrum in water showed maxima at 228 and 240

### 3. Synthesis of 1-((4-hydroxyphenyl)amino)ethylidene-1,1-diphosphonic acid

A solution of tetraethyl 1-((4-methoxyphenyl)amino)ethylidene-1,1-diphosphonate (5.0 g; 11.76 mMol, obtained in 2 above) in acetonitrile (70 ml) was stirred under argon. Trimethylsilyl bromide (13 ml) was added and the mixture refluxed for 8 hours. The solution was evaporated under reduced pressure and methanol (50 ml) was added and the solution was stirred at room temperature for 4 hours and lyophilized. After the removal of methanol and traces of hydrobromic acid, the product was isolated by adsorbing the bisphosphonic acid moiety onto pre-washed basic ion exchange resin (100 g, Dowex-550 OH). The impurities were washed with water and the desired product was isolated by leaching with water-ammonia (1:1) mixture. The resulting solution was lyophilized and dried. The product weighed 240 mg and showed an NMR spectrum consistent with the structure.

Proton NMR spectrum in D₂O:

δ7.0 (doublet, Ar—H, 2-H);

δ6.7 (doublet, Ar—H, 2 H) and

δ1.3 (triplet, CH₃, 3H).

### 4. Synthesis of 4-O-glucopyranosyloxyacetophenone

A solution of 4-hydroxyacetophenone (2.28 g; 16.8 mMol) and tetra-O-benzyl glucopyranose (5 g; 9.2 mMol) in dichloromethane (75 ml) was stirred under argon with molecular sieves (10 g). To this mixture was introduced boron trifluoride etherate (1 ml; 8.4 mMol). The mixture was stirred and monitored by TLC using 30% ethyl acetate in hexane. The reaction was essentially complete in 6 hours and the product was isolated by pouring 100 ml water and extracting into dichloromethane (100 ml). The combined organic layer was washed once with saturated sodium bicarbonate solution and followed by water. After evaporating the solvent, silica gel chromatography afforded the desired glycosylated material (2.2 g).

The structure of the compound was confirmed by proton NMR spectrum.

In CDCl₃:

δ7.9 (doublet, aryl, 2H);

δ7-7.5 (multiplet, aryl, 22H),

δ5.5-3.3 (multiplets, benzyl and sugar-H, 15H) and

δ2.6 (singlet, acetyl, 3H).

### 5. Synthesis of oximino-4-O-(2′,3′,4′,6′-tetra-O-benzyl)glucopyranosyloxy-phenylacetophenone.

To methanol (30 ml) and hydroxylamine hydrochloride (1 g, large excess) was added 25% sodium hydroxide (2.3 ml). The mixture was refluxed for 5 minutes. A solution of 4-O-(2′,3′,4′,6′-tetra-O-benzyl)glucopyranosyloxyphenylacetophenone (1.87 g) in acetonitrile (30 ml) was added. The mixture was refluxed for 6 hours. The progress of the reaction was monitored by TLC (30% ethyl acetate in hexane). The reaction mixture was evaporated under reduced pressure and water (200 ml) was added. The mixture was extracted with chloroform (3×150 ml). The combined organic portion was washed once with water, evaporated and chromatographed over silica gel. The desired oxime (1.8 g) was obtained and was used without any characterization.

### 6. Synthesis of tetraethyl 1-{(4-O-(2′,3′,4′,6′-tetra-O-benzylglucopyranosyl)oxyphenyl)amino}ethylidene-1,1-diphosphonate

A solution of oximino-4-O-(2′,3′,4′,6′-tetra-O-benzyl)glucopyranosyloxy-phenylacetophenone (1.8 g) in dichloromethane (70 ml) was cooled to 4° C. and blanketed with argon. Triethyl phosphite (1 ml) was added, followed by phosphorus oxychloride (0.5 ml). The reaction mixture was allowed to warm to room temperature and stirred for 14 hours. The reaction mixture was quenched with 1:1 ammonium hydroxide: water (100 ml) in a separator funnel and extracted with ethyl acetate (3×100 ml). The combined organic portion was washed with water (250 ml) once and dried over magnesium sulfate. The purification of the product by column flash chromatography over silica gel and mixtures of dichloromethane and ethyl acetate afforded pure tetraethyl 1-{(4-O-(2′,3′,4′,6′-tetra-O-benzylglucopyranosyl)oxyphenyl)amino}ethylidene-1,1-diphosphonate (1.36 g).

The product was characterized by proton NMR spectrum.

In CDCl₃ δ6.9-7.5 (complex, aryl-H, 24H);

δ3.5-5.4 (multiplets, benzylic-CH2 & O—CH₂—CH₃ & sugar-H, 23H);

δ1.6 (triplet, CH₃, 3H) and

δ1.3 (overlapping triplets, OCH₂CH₃, 12H).

### 7. Synthesis of 1{((4-O-glucopyranosyl)oxyphenyl)amino}ethylidene-1,1-diphosphonic acid

A solution of tetraethyl 1-{(4-O-(2′,3′,4′,6′-tetra-O-benzylglucopyranosyl)oxyphenyl)amino}ethylidene-1,1-diphosphonate (920 mg, 1 mmol) in dry acetonitrile (20 ml) under argon was stirred and cooled to 4° C. Trimethylsilyl bromide (3.6 g) was added and the mixture stirred at room temperature for 24 hours. The solvents were removed under high vacuum and more volatiles removed by lyophilization. Methanol-water (10 ml, 1:1) was added and stirred at 4° C. for 20 minutes and lyophilized again. The gum was dissolved in methanol and precipitated with dichloromethane.

The precipitate was centrifuged and removed from the dichloromethane layer. The precipitate was neutralized with 1 mg/ml of sodium hydroxide till neutral (pH 6 to 7), requiring 13.3 mg of sodium hydroxide. The sodium salt of the titled bisphosphonic acid was lyophilized and a gum weighing 97 mg was obtained.

The proton NMR spectrum of 1-{((4-O-glucopyranosyl)oxyphenyl)amino}-ethylidene-1,1-diphosphonic acid was recorded as its sodium salt.

In D₂O δ7.2 (doublet, aromatic-H, 2H)

δ7.8 (doublet, aromatic-H, 2H),

δ3.2 to 4.5 (broad multiplets, sugar-H, 7H) and

δ1.2 (triplet, CH₃, 3H).

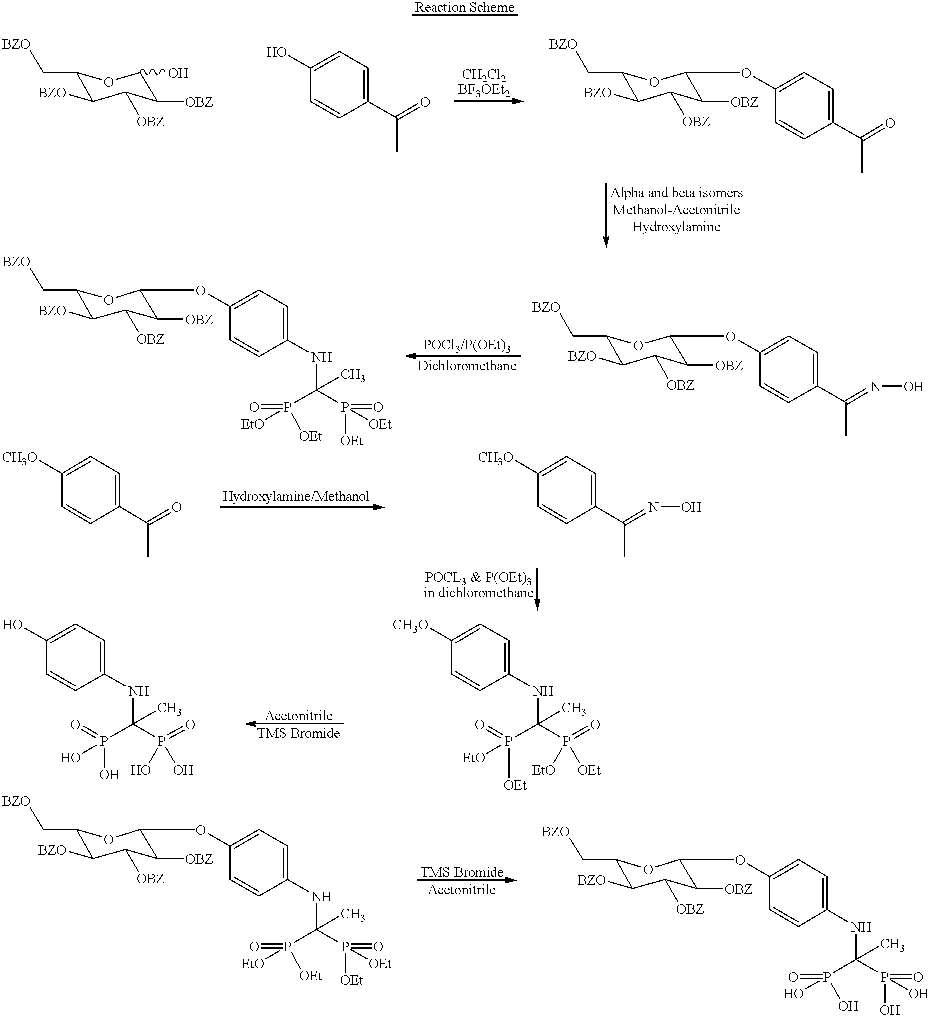

### EXAMPLE 5

### Preparation Of Condensation Products Of D-Glucose And Alendronate

### Experiment 1) Preparation of (4-amino-1-hydroxybutylidene)-1,1-bisphosphonic acid monosodium salt (Alendronate salt)

A 250 ml, three-necked, round-bottomed flask was fitted with a magnetic stirrer, an addition funnel, and a reflux condenser. The system was flushed with argon and charged with 4-aminobutyric acid (20 g, 0.19 mol), phosphorous acid (16 g, 0.19 mol) and methanesulphonic acid (80 ml). The mixture was heated to 65° C., and PCl₃ (35 ml, 0.40 mol) was added dropwise with stirring over a period of 25 min. The resulting mixture was maintained at 65° C. for 16 h. The clear, colorless solution was cooled to room temperature and quenched into 200 ml cold water (0-4° C.), with vigorous stirring. The reaction flask was rinsed with an additional 100 ml of water and the combined solution refluxed for 5 h. The solution was cooled to room temperature, the pH adjusted to 4.4 with 50% NaOH (ca.120 ml) and the resulting suspension left to cool in the fridge for to 2 days. The white crystalline product was collected by filtration, washed with cold water (100 ml) and 95% ethanol (100 ml) and dried in vacuo at room temperature, yielding 55.63 g (88% yield).

### Experiment 2) Preparation of (4-amino-1-hydroxybutylidene)-1,1-bisphosphonic acid (Alendronate):

A 250 ml, three-necked, round bottomed flask was fitted with a magnetic stirrer, an addition funnel, and a reflux condenser. The system was flushed with argon and charged with 4-aminobutyric acid (20 g, 0.19 mol), phosphorous acid (16 g, 0.19 mol) and methanesulphonic acid (80 ml). The mixture was heated to 65° C., PCl₃ (35 ml, 0.40 mol) added dropwise, with stirring, over a period of 25 min, and the mixture maintained at 65° C. for 24 h. The clear, colorless solution was cooled to room temperature and quenched into 200ml cold water (0-4° C.), with vigorous stirring. The reaction flask was rinsed with an additional 100 ml of water and the combined solution refluxed for 5 h. After cooling the reaction mixture to room temperature, the water and excess HCl were evaporated in vacuo (40° C.). The resulting thick oil was diluted with distilled water (140 ml) and cooled to 0° C., the pH adjusted to 0.55 with 50% NaOH (ca. 20 ml) and the crystalline product precipitated was collected by filtration, washed with cold water (100 ml) and absolute ethanol (100 ml) and dried in vacuo at room temperature, yielding 42.18 g (87.3% yield) of white crystalline alendronate.

¹H NMR (D₂O)

δ3.02 (2H, m),

δ1.99 (4H, m);

³¹P NMR (H₃PO₄, D₂O )

δ19.04

MS m/z 248 (86%, M—H), 227, 209. 191,173,166,159,155. 137(50%), 124, 113, 91(100%).

### Experiment 3) Preparation of N-Glucopyranosyl alendronate triethylamine salt

### a) At pH6

4-Amino-1-hydroxybutylidene-1,1-bisphosphonic acid (obtained from Experiment 2; 2 g, 8.02 mmol) was dissolved in distilled water (30 ml). The mixture was cooled in an ice-water bath and triethylamine was added dropwise, with stirring, until all 4-amino-1-hydroxybutylidene bisphosphonic acid went into solution, and the pH was 6. The water was then evaporated under vacuum at 35° C., yielding a thick, nearly colorless syrup. After the residue was taken up in methanol (30 ml), glucose (1.00 g, 5.55 mmol) was added and the mixture was heated under reflux on an oil bath at (70° C.), with stirring, until the glucose went into solution. After this, the mixture was allowed to stand, with stirring, at room temperature for 2 days. The mixture was then evaporated to dryness under vacuum, yielding a yellow foam of N-glucopyranosyl alendronate triethylamine salt as a mixture of α- and β-anomers (4.63 g, 70% yield).

¹H NMR (D₂O)

δ5.32 (0.5 H, d, J═6.2Hz, α-D-glucopyranosyl-H1β),

δ4.59 (0.5 H, d J═9.4 Hz, β-D-glucopyranosyl-H1α),

δ4.15-3.25 (6H, m, D-glucosyl-H),

δ3.25 (24 H. q, J═8 Hz, TEA-CH₂—),

δ3.20 (1H, m, —HNCH₂—CH₂—CH₂—),

δ2.95 (1H, m, —HNCH₂—CH₂—CH₂—),

δ1.80 (4H, m, HNCH₂—CH₂—CH₂—)

δ1.36 (36H, t, J═8 Hz, TEA-CH₃);

³¹P NMR (H3PO₄, D₂O)

δ18.07, 18.22.

### b) At pH 7.8

4-Amino-1-hydroxybutylidene-1,1-bisphosphonic acid (obtained from Experiment 2) (2 g, 8.02 mmol) was dissolved in distilled water (30 ml). The mixture was cooled in an ice-water bath and triethylamine (ca. 3.8 g) was added dropwise, with stirring, until all 4-amino-1-hydroxybutylidene-1,1-bisphosphonic acid went into solution and the pH was 7.8. The water was then evaporated under vacuum at 35° C., yielding a thick, nearly colorless syrup. After the residue had been taken up in methanol (30 ml), glucose (1.00 g, 5.55 mmol) was added and the mixture was heated under reflux on an oil bath at 70° C., with stirring, until all glucose went into solution. After this, the mixture was allowed to stir at room temperature for 2 days. The mixture was then evaporated to dryness under vacuum, yielding a yellow foam of N-glucopyranosyl alendronate triethylamine salt as a mixture of α- and β- anomers (4.86 g, 74% yield).

### Experiment 4) Large Scale Preparation

4-Amino-1-hydroxybutylidene-1,1-bisphosphonic acid (obtained from Experiment 2) (16 g, 64.22 mmol) was dissolved in distilled water (60 ml). The mixture was cooled in an ice-water bath and triethylamine (ca.26 ml) added dropwise, with stirring, until all 4-amino-1-hydroxybutylidene-1,1-bisphosphonic acid went into solution and the pH was 7.8. The water was then evaporated under vacuum at 35° C., yielding a thick, nearly colorless syrup. After the residue had been taken up in methanol (250 ml), glucose (8 g, 44.4 mmol) was added and the mixture was heated under reflux on an oil bath at 70° C., with stirring, until all glucose went into solution. After this, the mixture was allowed to stir at room temperature for 2 days. The mixture was evaporated to dryness under vacuum yielding a thick yellow oil N-glucopyranosyl alendronate triethylamine salt (41.56 g, 79% yield).

### Experiment 5) Preparation of N-glucopyranosyl Alendronate Sodium Salt

The yellow foam of N-glucopyranosyl alendronate triethylamine salt (4.86 g, 5,96 mmol) was dissolved in distilled water (35 ml) and loaded on to an Amberlite resin CG-120 (Na+ form) column and eluted with distilled water (300 ml). The appropriate fractions were collected, combined (according to the yellow color) and evaporated to dryness, yielding a yellow foam. This was then dissolved in water (25 ml) and lyophilized to give the title compound as a yellow solid (2.87 g, 100% yield), identified as a mixture of α- and β-anomers.

¹H NMR (D₂O)

δ5.35 (0.5 H, d, J═6.2 Hz, α-D-glucopyranosyl-H 1β),

δ4.59 (0.5 H, d J═9.4 Hz, β-D-glucopyranosyl-H1α),

δ4.15-3.25 (6H, m, D-glucopyranosyl-H),

δ3.20 (1H, m, —HNCH₂—CH₂—CH₂—),

δ2.95 (1H, m, —HNCH₂—CH₂—CH₂—),

δ1.90 (4H, m, HNCH₂—CH₂—CH₂—); p ³¹P NMR (H₃PO₄, D₂O)

δ18.49, 18.40;

MS m/z 499 (M, 10%), 456 M−2Na+3H, 22%), 434 (M−3Na+4H, 80%), 412 (M−4Na+5H, 90%), 380, 374, 358, 294, 272, 250, 168.

### b) At pH 7.8 with excess D-glucose

4-Amino-1-hydroxybutylidene-1,1-bisphosphonic acid (obtained from Experiment 2; 4.71 g, 9.44 mmol) was dissolved in distilled water (40 ml). The mixture was cooled in an ice-water bath and triethylamine added dropwise with stirring until all 4-amino-1-hydroxybutylidene-1,1-bisphosphonic acid went into solution and the pH was 7.8. The water was then evaporated under vacuum at 35° C., yielding a thick, nearly colorless syrup. After the residue had been taken up in methanol (30 ml), glucose (2.04 g, 11.32 mmol) was added and the mixture was heated under reflux on an oil bath at 70° C., with stirring, until all glucose went into solution. After this, the mixture was allowed to stir at room temperature for 2 days. The mixture was then evaporated to dryness under vacuum yielding a yellow foam of N-glucopyranosyl alendronate triethylamine salt. This was dissolved in distilled water (35 ml) and loaded onto an Amberlite resin CG-120 (Na+) column (5×25 cm) and eluted with distilled water (300 ml). The appropriate fractions were collected, combined (according to the yellow color), and evaporated to dryness yielding a yellow solid. This was then dissolved in water (35 ml) and lyophilized to give the title compound as a yellow solid (7.72 g, 82% yield).

¹H NMR (D₂O)

δ5.35 (0.5 H, d, J═6.2 Hz, α-D-glucopyranosyl-H1β),

δ4.55 (0.5 H, d J═9.3 Hz, β-D-glucopyranosyl-H1α),

δ4.20-3.25 (6H, m, D-glucosyl-H),

δ3.20 (1H, m, —HNCH₂—CH₂—CH₂—),

δ2.90 (1H, m, —HNCH₂—CH₂—CH₂—),

δ1.90 (4H, m, HNCH₂—CH₂—CH₂—);

³¹P NMR (H₃PO₄, D₂O)

δ18.49,18.40;

MS m/z 499 (M, 10%), 456 (M−2Na+3H, 22%),434 (M−3Na+4H, 80%), 412 (M−4Na+5H, 90%), 380, 374, 358, 294, 272, 250, 168.

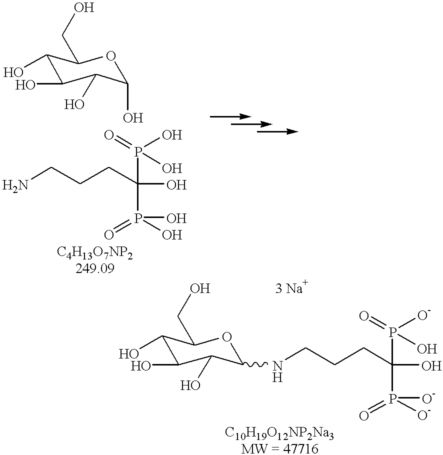

### Biological Activity

### Assay of Bone Resorption in vitro

Bone resorption in vitro was assessed by a modification of the “bone slice assay” (Walsh et al., (1991), Application of Reflected Light Microscopy to Identify and Quantitate Resorption by Isolated Osteoclasts, Journal of Bone and Mineral Research, Vol. 6: 661-671). Sterile, devitalized discs of dentine (diameter 4 mm) where prepared and placed in a 96 well tissue culture plate, one disc per well, and pre-wet overnight in α-MEM tissue culture medium supplemented with 10% fetal calf serum. The femora and tibiae where dissected from 18 day chick embryos and transferred into α-MEM culture medium supplemented with 10% fetal calf serum.

The bones were finely minced using a sharp scalpel and the resulting suspension was triturated using a sterile pipette and transferred to a sterile plastic tube. The suspension was allowed to stand for one minute so that fragments of bone and other debris settled and the upper layer containing a bone cell suspension, including osteoclasts and other cell types, was harvested.

Aliquots of this bone cell suspension were added to the culture wells containing the devitalized dentine discs. Cells where allowed to settle for between 2 and 24 hours. The medium was then removed and new medium containing the test reagents was added. Cells where cultured for periods of time up to 72 hours after which time the dentine discs were washed in PBS at 37° C., fixed in 4% glutaraldehyde in 0.2 M sodium cacodylate, and stained for 5 minutes in 1% (w/v) toluidine blue in 0.5% disodium tetraborate. Excess stain was removed by washing in 70% ethanol for one minute. The dentine discs were then washed in tap water and air-dried.

Resorption lacunae where identified and quantitated using an Olympus BH2 microscope fitted for incident light microscopy. The plan area of resorption was determined by point counting using a ×10 objective and a drawing tube. The total area of each disc was analyzed.

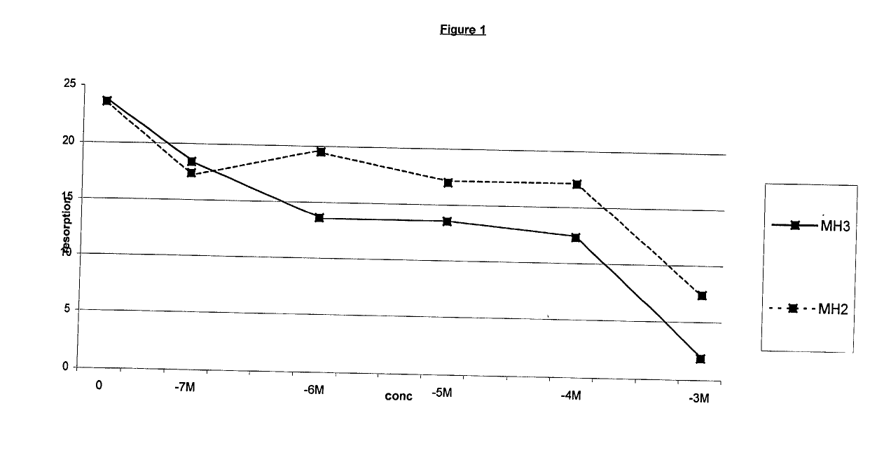

In the accompanying Figures, both cell number and resorption are as calculated from the foregoing. In FIG. 1, the glycosylated and non-glycosylated product of Example 4 are compared. From the Figure, it can be seen that MH2 (the non-glycosylated product—1-ethylidene-1,1-bisphosphonic acid) has substantially less effect than MH3 (the glycosylated product-1-((4-O-glucopyranosyl)oxyphenyl)aminoethylidene-1,1-bisphosphonic acid).

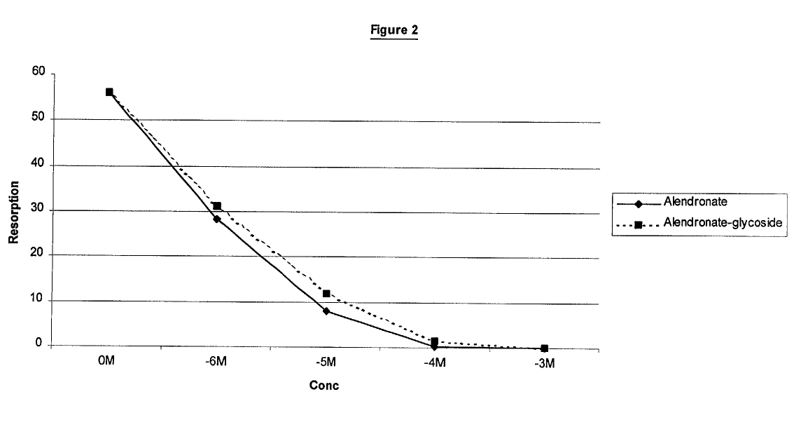

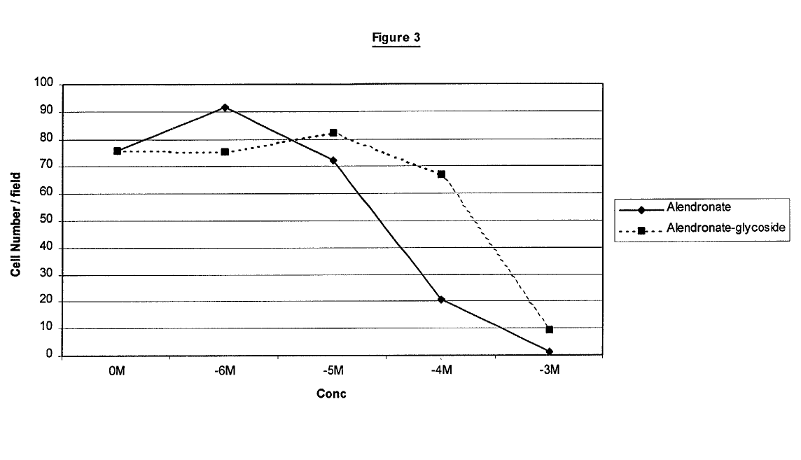

Likewise, FIGS. 2 and 3 compare the effects of glycosylated and non-glycosylated alendronate, as prepared in Example 5.

FIG. 2 shows that both the alendronate and its glucoside have similar effects on resorption, while FIG. 3 shows a clear effect on toxicity, with the glucoside exhibiting substantially no toxicity up to concentrations of 10⁻⁴M whereas, at this concentration, alendronate kills the majority of cells.

From the foregoing description, one skilled in the art can easily ascertain the essential characteristics of this invention, and without departing from the spirit and scope thereof, can make various changes and modifications of the invention to adapt it to various usages and conditions without undue experimentation. All patents, patent applications and publications cited herein are incorporated by reference in their entirety.

What is claimed is:

1. A glycoside or orthoester glycoside derivative of a therapeutically useful bisphosphonate compound, or salt, ester or pro-drug thereof.

2. The compound of claim 1, comprising a P—C—P linkage.

3. A compound of formula (I): 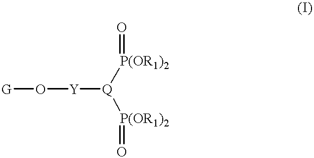
wherein G is a straight or branched chain glycosidic residue containing 1-20 glycosidic units per residue, or G is an orthoester glycoside moiety of the Formula (II): 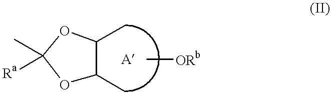
wherein A′ is a glycofuranosyl or glycopyranosyl ring;
R^{a} is hydrogen;
R^{b} is hydrogen or a straight or branched chain glycosidic residue containing 1-20 glycosidic units per residue;
each R₁, which may be the same or different, is hydrogen, alkyl, aryl, benzyl or alkali metal cation, or two —OR₁ groups, on the same phosphorus atom, taken together with —CH₂)₂—, —CH₂)₃—, or —CH₂C(CH₃)₂CH₂—, form a heterocyclic ring containing one phosphorus, two oxygens and two or three carbons;
Q is selected from the group consisting of:
(1) —CH—, —CNH₂—, —COH—, —CCl—, —CF— or —C-alkyl—;
(2) —C(R₄)(R₅)(CH₂)ₘ[C(R₆)(R₇)]ₙ—;
wherein n is 0 or 1 and m is an integer from 1 to 8;
R₄ and R₆, which may be the same or different, are hydrogen, —SO₃H, a lower aliphatic group which may optionally contain one or more heteroatoms and which contains at least one —SO₃H group or a covalent bond to Y,
R₅ and R₇, which may be the same or different, are hydrogen, —OH, —NH₂, —NHMe, —NMe₂, —SO₃H, substituted alkyl or a covalent bond to Y; or
R₄ and R₅ taken together with the atom to which they are bound form a carbonyl, thiocarbonyl, or ═NOH; and
R₆ and R₇ taken together with the atom to which they are bound form a carbonyl, thiocarbonyl, or ═NOH;
(3) [chemical structure]
wherein R₈ and R₉, which may be the same or different, are:
(a) a covalent bond to Y, —NO₂ or —NH₂;
(b) —SR₁₁ wherein R₁₁ is hydrogen, alkyl, phenyl, acyl, benzoyl, aralkyl, halogen, allyl or a covalent bond to Y;
(c) —OR₁₀ wherein R₁₀ is hydrogen, alkyl, allyl, acyl, benzoyl, aralkyl or a covalent bond to Y;
(d) —CH₂)ₚCO₂A′, wherein A′ is hydrogen, alkyl or a covalent bond to Y;
(e) —(CH₂)ₚCH₂OR₁₀ wherein R₁₀ is defined as above;
(f) —CH₂NHR₁₂ wherein R₁₂ is hydrogen, alkyl, phenyl, acyl, benzoyl, aralkyl or a covalent bond to Y;
(g) —CH₂N(R₁₂)(R₁₃) wherein R₁₂ and R₁₃ can be the same or different and are hydrogen, alkyl, phenyl, acyl, benzoyl, aralkyl or a covalent bond to Y;
a is 1 or 2;
b is 1 to 4;
R^{c} and R^{d}, which may be the same or different in each instance, are hydrogen or alkyl;
with the proviso that when the ring containing R₈ is a pyridine ring, b is 1 to 3;
p is 1 to 5;
with the proviso that only one of A′, R₄, R₅, R₆, R₇, R₈, R₉, R₁₀, R₁₁, R₁₂ and R₁₃ is a covalent bond to Y;
Y is chosen from the group consisting of optionally substituted C₁₋₁₀ alkylene, aryl, heteroaryl, heterocyclo, a steroidal hormone, a compound exhibiting oestrogenic activity or a prostaglandin; and
pharmaceutically acceptable salts or esters thereof.

4. The compound of claim 3, comprising a P—C—P linkage.

5. A compound of Formula (I′): 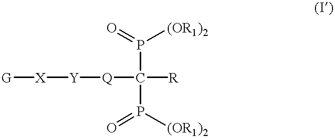
wherein each OR₁ is the same or different and is OH or a hydrolysable group, or two OR₁ groups on the same phosphorus atom form a substituted or unsubstituted 5, 6 or 7 membered heterocyclic ring;
R is H, alkyl or halogen or is a group —O—G, —S—G, —NH—G or —NR₁₂—G;
each G is the same or different and is hydrogen or a straight or branched chain glycosidic residue or glycosidic orthoester residue, or amino derivative thereof, provided that at least one group G is a glycosidic residue or glycosidic orthoester residue,
X is O, S, NH or NR₁₂;
each R₁₂ is the same or different and is hydrogen, alkyl, phenyl, acyl, benzoyl or aralkyl;
Q is an optionally substituted alkylene or alkenylene group or is an optionally substituted alkylene containing at least one O, S or NH or is O, S or NH, or is a direct bond to Y;
Y represents a binary or tertiary alkyl-substituted amine, C₁₋₁₀ alkylene, aryl, heteroaryl, heterocyclyl, a steroidal hormone, a group exhibiting oestrogenic activity or a prostaglandin, said group Y being optionally substituted;
provided that, in —X—Y—Q— there is no direct bond between one O, S or N atom and another O, S or N,
and pharmaceutically acceptable salts, esters and pro-drugs thereof.

6. The compound of claim 5, wherein OR₁ is a hydrolysable group, and R₁ represents an optionally substituted, alkyl, aryl, or aralkyl group.

7. The compound of any of claim 5, wherein R₁ represents an alkali metal cation.

8. The compound of claim 5, wherein two OR₁ groups on the same phosphorus atom form a substituted or unsubstituted 5, 6 or 7 membered heterocyclic ring together with the phosphorus to which they are attached.

9. The compound of claim 5, wherein R is methyl or ethyl.

10. The compound of claim 5, wherein R is halogen.

11. The compound of claim 5, wherein R is H.

12. The compound of claim 5, wherein R is Cl.

13. The compound of claim 5 which is a glycosidic derivative comprising a glycosidic residue containing 1-20 glycosidic units.

14. The compound of claim 13, wherein said glycosidic residue contains only one glycosidic unit.

15. The compound of claim 14, wherein the unit is a glucosyl residue.

16. The compound of claim 5 which is a glycosidic orthoester derivative comprising a glycosidic orthoester residue having the Formula (II): 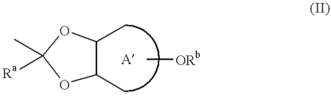
wherein A′ represents a glycofuranosyl or glycopyranosyl ring or amino derivative thereof;
R^{a} is hydrogen, C₁₋₄ alkyl, C₇₋₁₀ aralkyl, phenyl; or phenyl substituted by chloro, fluoro, bromo, iodo, C₁₋₄ alkyl or C₁₋₄ alkoxy; or naphthyl; and R^{b} is hydrogen or a straight or branched chain glycosidic residue containing 1-20 glycosidic units per residue.

17. The compound of claim 5, wherein Q is an alkylene group containing 1 to 7 carbons in the chain.

18. The compound of claim 5, wherein Q is O, S or NH.

19. The compound of claim 5, wherein Y represents an amine, the nitrogen of which is directly linked to Q.

20. The compound of claim 19, wherein Y is substituted by one or two alkyl groups.

21. The compound of claim 5, wherein Y is a C₁₋₁₀ alkylene group.

22. The compound of claim 21, wherein Y is methyl or ethyl.

23. The compound of claim 5, wherein Y is aryl.

24. The compound of claim 23, wherein Y is phenyl, chlorophenyl or naphthyl.

25. The compound of claim 5, wherein Y represents a heteroaryl group.

26. The compound of claim 2, wherein Y is pyrazolyl, imidazolyl or pyridinyl.

27. The compound of claim 5, wherein Y is a heterocyclic group.

28. The compound of claim 27, wherein Y is pyrrolidinyl or pyrimidinyl.

29. The compound of claim 5, wherein Y is a steroidal hormone residue.

30. The compound of claim 5, wherein at least 3 R₁ groups are hydrogen atoms.

31. The compound of claim 5, wherein the residue 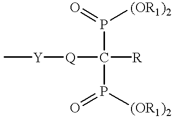
is derived from:
(4-amino-1-hydroxybutylidene)bisphosphonate;
(1-hydroxy-3-(1-pyrrolidinyl)propylidene)bisphosphonate;
(1-hydroxyethylidene)bisphosphonate;
(1-hydroxy-3-(methylpentylamino)propylidene)bisphosphonate;
((cycloheptylamino)methylene)bisphosphonate;
(6-amino-1-hydroxyhexylidene)bisphosphonate;
(3-(dimethylamino)-1-hydroxypropylidene)bisphosphonate;
(3-amino-1-hydroxypropylidene)bisphosphonate;
(1-hydroxy-2-(3-pyridinyl)ethylidene)bisphosphonate;
((4-chlorophenylthio)methylene)bisphosphonate;
(1-hydroxy-2-imidazo(3,2a)pyridin-3-ylethylidene)bisphosphonate; or
(1-hydroxy-2-(1H-imidazol-1-yl)ethylidene)bisphosphonate.

32. The compound of claim 5, having the Formula (XI): 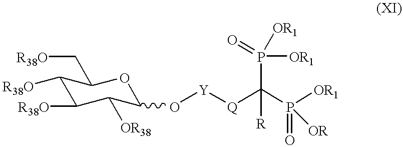
wherein R₃₈ is selected from the group consisting of hydrogen, acetyl, benzoyl, nicotinoyl, benzyl, methyl and phenyl; and each R, R₁, Y and Q is as defined; and pharmaceutically acceptable salts and esters thereof.

33. The compound of claim 32, wherein each R₁ is selected from the group consisting of hydrogen, alkyl, benzyl and phenyl.

34. N-glucopyranosyl alendronate.

35. 1-{((4-O-glucopyranosyl)oxyphenyl)amino}ethylidene-1,1-diphosphonic acid.

36. 4-O-(1′-glucopyranosyl)-4-hydroxybutane-1,1-bisphosphonic acid.

37. A pharmaceutical composition comprising the compound of claim 1, together with a pharmaceutically acceptable carrier therefor.

38. A method of treatment of a condition treatable by administration of a bisphosphonate compound, comprising administration of a non-toxic, efficacious amount of the compound of claim 1 to a patient in need thereof.

39. The composition of claim 37, for oral administration.

40. The composition of claim 37, which is a transdermal patch.

41. A method of preparing a compound of Formula (XI): 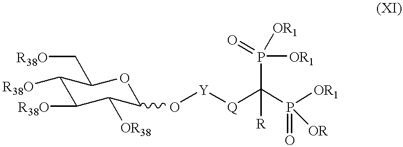
wherein each OR₁ is the same or different and is OH or a hydrolysable group, or two OR₁ groups on the same phosphorus atom form a substituted or unsubstituted 5, 6 or 7 membered heterocyclic ring;
R is H, alkyl or halogen or is a group —O—G, —S—G, —NH—G or —NR₁₂—G;
G is hydrogen or a straight or branched chain glycosidic residue or glycosidic orthoester residue, or amino derivative thereof, provided that at least one group G is a glycosidic residue or glycosidic orthoester residue,
R₁₂ is hydrogen, alkyl, phenyl, acyl, benzoyl or aralkyl;
Q is an optionally substituted alkylene or alkenylene group or is an optionally substituted alkylene containing at least one O, S or NH or is O, S or NH; Y represents a binary or tertiary alkyl-substituted amine, C₁₋₁₀ alkylene, aryl, heteroaryl, heterocyclyl, a steroidal hormone, a group exhibiting oestrogenic activity or a prostaglandin, said group Y being optionally substituted; provided that, in —O—Y—Q— there is no direct bond between one O, S or N atom and another O, S or N,
R₃₈ is selected from the group consisting of hydrogen, acetyl, benzoyl, nicotinoyl, benzyl, methyl and phenyl; which method comprises:
(a) reacting a glycoside having the Formula (XII): 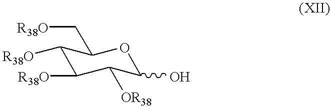
wherein R₃₈ is other than hydrogen; with a group of formula Hal-Y—Q—X₃, wherein Hal and X₃ individually represent halogen, and is preferably a 1,3-dihaloalkane, in the presence of a strong base in an aprotic solvent to give a compound of Formula (XIII): 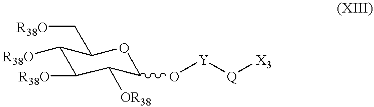
wherein X₃ is as defined; and
(b) reacting the compound of Formula (XIII) with a compound of the Formula (XIV): 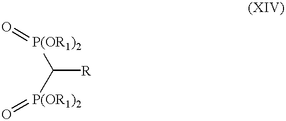
wherein R and R₁ are as defined, in the presence of a strong base in an aprotic solvent to give a compound of Formula (XI), wherein R₁ and R₃₈ are other than hydrogen.

42. A process for the preparation of a compound of formula (XX): 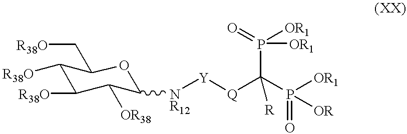
wherein each OR₁ is the same or different and is OH or a hydrolysable group, or two OR₁ groups on the same phosphorus atom form a substituted or unsubstituted 5, 6 or 7 membered heterocyclic ring;
R is H, alkyl or halogen or is a group —O—G, —S—G, —NH—G or —NR₁₂—G;
G is hydrogen or a straight or branched chain glycosidic residue or glycosidic orthoester residue, or amino derivative thereof, provided that at least one group G is a glycosidic residue or glycosidic orthoester residue,
R₁₂ is hydrogen, alkyl, phenyl, acyl, benzoyl or aralkyl;
Q is an optionally substituted alkylene or alkenylene group or is an optionally substituted alkylene containing at least one O, S or NH or is O, S or NH;
Y represents a binary or tertiary alkyl-substituted amine, C₁₋₁₀ alkylene, aryl, heteroaryl, heterocyclyl, a steroidal hormone, a group exhibiting oestrogenic activity or a prostaglandin, said group Y being optionally substituted; provided that, in —N—Y—Q— there is no direct bond between one O, S or N atom and another O, S or N,
R₃₈ is selected from the group consisting of hydrogen, acetyl, benzoyl, nicotinoyl, benzyl, methyl and phenyl;
said method comprising dissolving a compound of formula (XXI): 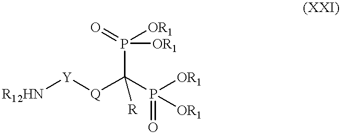
in an aqueous medium together with sufficient of a trialkylamine compound to form a salt thereof;
removing the water;
dissolving the salt in a water miscible, organic solvent;
adding a compound of formula (XII): 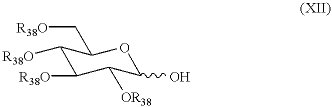
and maintaining the resulting mix under such conditions as to form a trialkylamine salt of the compound of formula (XX).
